SEQUENCE LISTING (12 sequences)

SEQ ID NO: 1 - RNA, 19 nt, Artificial
  Synthetic primer
uagguaguuucauguuguu

SEQ ID NO: 2 - protein, 590 aa, Homo sapiens
MSRQSSVSFRSGGSRSFSTASAITPSVSRTSFTSVSRSGGGGGGGFGRVSLAGACGVGGYGSRSLYNLGGSKRISISTSGGSFRNRFGAGAGGGYGFGGGAGSGFGFGGGAGGGFGLGGGAGFGGGFGGPGFPVCPPGGIQEVTVNQSLLTPLNLQIDPSIQRVRTEEREQIKTLNNKFASFIDKVRFLEQQNKVLDTKWTLLQEQGTKTVRQNLEPLFEQYINNLRRQLDSIVGERGRLDSELRNMQDLVEDFKNKYEDEINKRTTAENEFVMLKKDVDAAYMNKVELEAKVDALMDEINFMKMFFDAELSQMQTHVSDTSVVLSMDNNRNLDLDSIIAEVKAQYEEIANRSRTEAESWYQTKYEELQQTAGRHGDDLRNTKHEISEMNRMIQRLRAEIDNVKKQCANLQNAIADAEQRGELALKDARNKLAELEEALQKAKQDMARLLREYQELMNTKLALDVEIATYRKLLEGEECRLSGEGVGPVNISVVTSSVSSGYGSGSGYGGGLGGGLGGGLGGGLAGGSSGSYYSSSSGGVGLGGGLSVGGSGFSASSGRGLGVGFGSGGGSSSSVKFVSTTSSSRKSFKS

SEQ ID NO: 3 - protein, 724 aa, Homo sapiens
MSAEGYQYRALYDYKKEREEDIDLHLGDILTVNKGSLVALGFSDGQEARPEEIGWLNGYNETTGERGDFPGTYVEYIGRKKISPPTPKPRPPRPLPVAPGSSKTEADVEQQALTLPDLAEQFAPPDIAPPLLIKLVEAIEKKGLECSTLYRTQSSSNLAELRQLLDCDTPSVDLEMIDVHVLADAFKRYLLDLPNPVIPAAVYSEMISLAPEVQSSEEYIQLLKKLIRSPSIPHQYWLTLQYLLKHFFKLSQTSSKNLLNARVLSEIFSPMLFRFSAASSDNTENLIKVIEILISTEWNERQPAPALPPKPPKPTTVANNGMNNNMSLQDAEWYWGDISREEVNEKLRDTADGTFLVRDASTKMHGDYTLTLRKGGNNKLIKIFHRDGKYGFSDPLTFSSVVELINHYRNESLAQYNPKLDVKLLYPVSKYQQDQVVKEDNIEAVGKKLHEYNTQFQEKSREYDRLYEEYTRTSQEIQMKRTAIEAFNETIKIFEEQCQTQERYSKEYIEKFKREGNEKEIQRIMHNYDKLKSRISEIIDSRRRLEEDLKKQAAEYREIDKRMNSIKPDLIQLRKTRDQYLMWLTQKGVRQKKLNEWLGNENTEDQYSLVEDDEDLPHHDEKTWNVGSSNRNKAENLLRGKRDGTFLVRESSKQGCYACSVVVDGEVKHCVINKTATGYGFAEPYNLYSSLKELVLHYQHTSLVQHNDSLNVTLAYPVYAQQRR

SEQ ID NO: 4 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 5 - DNA, 25 bp, Artificial
  Synthetic primer
tagtctagaggtcatagaacacatc

SEQ ID NO: 6 - DNA, 26 bp, Artificial
  Synthetic primer
tagtctagagacttggaggaagagac

SEQ ID NO: 7 - DNA, 26 bp, Artificial
  Synthetic primer
tagtctagacagcttcggaattcatc

SEQ ID NO: 8 - DNA, 27 bp, Artificial
  Synthetic primer
tagtctagagctactttattcagggag

SEQ ID NO: 9 - DNA, 24 bp, Artificial
  Synthetic primer
tagactagtgaacctgctgcaagt

SEQ ID NO: 10 - DNA, 24 bp, Artificial
  Synthetic primer
tagactagtatattataaaagcat

SEQ ID NO: 11 - DNA, 26 bp, Artificial
  Synthetic primer
gcatctagaacattcccttgatggtc

SEQ ID NO: 12 - DNA, 30 bp, Artificial
  Synthetic primer
ccgcatctagagtgacgtcattttattttc

(19) United States
(12) Patent Application Publication
(10) Pub. No.: US 2011/0275533 A1
(43) Pub. Date: Nov. 10, 2011

(54) Micro-RNAs as Markers for Tumor Progression

(75) Inventors: Rajyalakshmi Luthra, Missouri City, TX (US); Madan Luthra, Missouri City, TX (US); Constance Albarracin, Houston, TX (US)

(73) Assignee: THE BOARD OF REGENTS OF THE UNIVERSITY OF TEXAS SY STEM, Austin, TX (US)

(21) Appl. No.: 13/125,277

(22) PCT Filed: Oct. 21, 2009
(86) PCT No.: PCT/US2009/061522

§ 371 Date: Jul. 27, 2011

Related U.S. Application Data
(60) Provisional application No. 61/107,226, filed on Oct. 21, 2008.

(51) Int. Cl. C40B 30/04; C12Q 1/02; C12Q 1/68

(52) U.S. Cl. 506/9; 435/6.14; 435/6.1; 435/6.11; 435/29; 435/6.12

(57) ABSTRACT

Disclosed are methods for diagnosing cancer or dysplasia in a sample of tissue, involving testing cells of the sample to assess whether miR-196a expression in cells of the sample is greater than a reference, wherein a greater expression of miR-196a in cells of the sample compared to the reference is indicative of cancer or dysplasia. The cancer or dysplasia may be esophageal cancer or esophageal dysplasia. Also disclosed are method for diagnosing cancer or dysplasia in a sample of tissue, comprising testing cells of the sample to assess whether the expression of ANXA1, KRT5, SPRR2C, or S100A9 in cells of the sample is reduced compared to a reference control, wherein said reduced expression in cells of the sample compared to the reference control is indicative of cancer or dysplasia. Also disclosed are kits and methods involving kits for the diagnosis of cancer or dysplasia.

The present application claims the benefit of priority to U.S. Provisional Patent Application Ser. No. 61/107,226, filed on Oct. 21, 2008, which is hereby incorporated by reference in its entirety.

### BACKGROUND OF THE INVENTION

1. Field of the Invention

The present invention relates generally to the fields of molecular biology, oncology, and diagnostic medicine. More particularly, the present invention concerns methods for diagnosing esophageal cancer or esophageal dysplasia that involve assessing levels of miR-196a in cells of an esophageal lesion, or the expression of annexin A1 (ANXA1), keratin 5 (KRT5), small proline-rich protein 2C (SPRR2C), or S100 calcium-binding protein A9 (S100A9) in cells of an esophageal lesion.

2. Description of Related Art

Despite significant advances in therapeutics and diagnostics, cancer remains a major cause of morbidity and mortality in the U.S. For some cancers, despite these advances, the incidence is on the rise. The incidence of esophageal adenocarcinoma (EA) has increased by three-four fold in the past three decades exceeding all other type of cancers (Devesa et al., 2000; Shaheen et al., 2000). Patients with EA generally present with advanced stage and in spite of aggressive treatment the overall 5-year survival is 25% (Swisher et al., 1995). Barrett's esophagus (BE)/Barrett Metaplasia (BM) is a recognized precursor of EA with an intermediate step of dysplasia. At present, histologic assessment is the gold standard for risk assessment of dysplasia or EA in BE patients. Esophagogastroduodenoscopy (EGD) with four quadrant biopsies to detect dysplasia and early invasive adenocarcinoma is the only regular screening method currently recommended for patients with BE. However, low yield and high cost of EGD screening necessitates a search for better methods and biomarkers to identify high-risk patients. Markers like aneuploidy, tetraploidy, and p16 and p53 genetic abnormalities (loss of heterozygosity of chromosomes 9p and 17p, respectively) have been correlated with risk assessment (Reid et al., 2001; Reid et al., 2003). However, in spite of extensive studies these markers have been of limited clinical utility, warranting the exploration of novel biomarkers like microRNA in BE and its progression to dysplasia and carcinoma.

MicroRNAs (miRNAs) are small (16-29 nucleotides) RNA molecules that are part of non-coding evolutionary conserved class of endogenous riboregulators that alter the gene expression through target mRNA degradation or by reducing the translation of target mRNA by binding to their 3′ untranslated region (Tricoli and Jacobson, 2007). In cancer and metastasis, increasing numbers of miRNAs have been implicated in the deregulation of gene expression, thus establishing them as an important new class of oncogenes and tumor suppressors (Esquela-Kerscher and Slack, 2006).

Gene expression signature analysis, which involves the analysis of global gene expression at mRNA level is widely used for cancer detection and diagnosis and to study progression (Ramaswamy et al., 2001). The relatively recent identification of miRNAs as an additional level of posttranscriptional regulation of gene expression has shifted the focus to evaluating their value as diagnostic and prognostic markers in cancer. To date, every type of tumor analyzed has had a miRNA profile significantly different from that of the respective normal tissue (Calin and Croce, 2006). Further, a recent study showed that tissue-specific expression pattern of miRNAs also makes them valuable in the identification of tissue origin in unknown primary cancers (Rosenfeld et al., 2008). Extensive studies have identified the tumor suppressor or oncogenic role of miRNAs, whose expression or functions are deregulated during the neoplastic transformation of tissues (Chen, 2005).

miR-196a has been shown to have a defined biologic function in hind limb development by acting on HOXB and sonic hedgehog (Shh) signaling (Hornshetin et al., 2005) and in the pathogenesis of acute myeloid leukemia (Debernardi et al., 2007). Furthermore, recent studies have demonstrated that miR-196a levels allowed discrimination of normal pancreas from chronic pancreatitis and adenocarcinoma and that miR-196a levels inversely correlated with survival in pancreatic adenocarcinoma patients (Bloomston et al., 2007).

Global gene expression profiling studies revealed that several genes including ANXA1 show gradual decrease in expression during progression of BE to EA (Kimchi et al., 2005; Selaru et al., 2002). These genes also included some that are downregulated in tumors resistant to preoperative chemoradiation in our previous studies (Luthra, 2007; Luthra, 2006). SPRR2C belongs to the family of small proline-rich (SPRR) proteins that function as crosslinkers of epidermal differentiation complex (EDC) proteins (Patel et al., 2003). This family of genes is known to be upregulated during inflammation, infection, and epithelial barrier remodeling (Cabral et al., 2001) and is functionally involved in protecting the epithelia from environmental insults. KRT5 is a structural protein that belongs to the family of cytokeratins and is downregulated in EA (Sato et al., 2006). The precise role of these genes in the underlying pathogenesis of cancer remains unknown.

Thus, there is the need for greater understanding of gene expression and regulators of gene expression in cancer, such as esophageal cancer. A greater understanding of these factors can facilitate the identification of methods to diagnosis cancer and to identify risk factors for progression to cancer. Such methods can be applied to reduce the significant morbidity and mortality associated with cancer.

### SUMMARY OF THE INVENTION

The present invention in part concerns the finding that assessment of miRNA can be applied in diagnosing cancer or dysplasia. For example, the inventors have found that higher levels of miR-196a were observed in EA, Barrett's esophagus, and dysplastic lesions of the esophagus compared to normal squamous mucosa, thus indicating that miR-196a is a marker for progression to esophageal cancer. The highest levels of miR-196a were observed in EA. Levels of miR-196a were higher in dysplastic lesions of the esophagus compared to normal esophageal tissue, this indicating that miR-196a also has application as a marker for esophageal dysplasia. Regarding miR-196a, it has been shown to have a defined biologic function in hind limb development by acting on HOXB and sonic hedgehog (Shh) signaling (Hornstein et al., 2005) and in the pathogenesis of acute myeloid leukemia (Debernardi et al., 2007).

The invention is also in part based on the finding that miR-196a reduces the expression of certain genes that are believed to be causally associated in cancer. Non-limiting examples of such cancers include esophageal, breast, and endometrial cancer. For instance, the inventors have found reduced ANXA1 expression in breast cancers and endometrial cancer cells correlates with increased miR-196a levels, thus indicating that miR-196a is a marker for these cancer types.

Particular aspects of the present invention generally concern methods of diagnosing a cancer or a predisposition to cancer in a subject with a lesion, comprising obtaining information regarding whether expression of a microRNA (miRNA) in cells of the lesion is greater than a reference control. miRNAs are small (16-29 nucleotides) RNA molecules that are part of non-coding evolutionary conserved class of endogenous riboregulators that alter gene expression through target mRNA degradation or by reducing the translation of target mRNA by binding to their 3′ untranslated region.

Further aspects of the invention concern methods for diagnosing cancer or a predisposition to cancer in a subject with a lesion, involving determining whether the expression of an miRNA in cells of the lesion is increased compared to a reference control miR-196a expression. Determining level of expression of a species of miRNA can be by any method known to those of ordinary skill in the art, and examples of such methods are described in greater detail in the specification below.

The cancer that is diagnosed or to which a predisposition is diagnosed may be any type of cancer. For example, the cancer may be esophageal cancer, breast cancer, endometrial cancer, stomach cancer, intestinal cancer, rectal cancer, lung cancer, prostate cancer, ovarian cancer, brain cancer, liver cancer, cervical cancer, colon cancer, renal cancer, skin cancer, or head and neck cancer. In particular embodiments, the cancer is esophageal cancer, endometrial cancer, or breast cancer. In a specific embodiment, the cancer is esophageal cancer.

In particular embodiments, the miRNA is miR-196a (SEQ ID NO:1) or a nucleic acid sequence that comprises 10 or more contiguous nucleotides of SEQ ID NO:1. In particular embodiments, the miRNA comprises 11, 12, 13, 14, 15, 16, 17, 18, 19, 20, 21, 22 or more contiguous nucleotides of SEQ ID NO:1, or any range of nucleotides of SEQ ID NO:1 derivable therein. The methods set forth herein may further involve diagnosing the subject with cancer or a predisposition to cancer if the miR-196a expression in cells of the lesion is greater than the miR-196 expression in normal cells.

Particular aspects of the present invention generally concern methods for diagnosing cancer (such as EA) of the esophagus in a sample of tissue, comprising testing cells of the sample to assess whether miR-196a (SEQ ID NO:1) expression in cells of the sample is greater than a reference control miR-196a expression, wherein a greater expression of miR-196a in cells of the sample compared to the reference control is indicative of esophageal cancer. The sample of tissue may be obtained, for example, from an esophageal lesion.

Further aspects of the present invention concern methods for diagnosing dysplasia of the esophagus in a sample of suspected cancerous tissue that involve testing cells of the sample to assess whether miR-196a (SEQ ID NO:1) expression in cells of the sample is greater than a reference control miR-196a expression, wherein a greater expression of miR-196a in cells of the sample compared to the reference control is indicative of esophageal dysplasia.

The methods set forth herein may or may not involve testing a “reference control” to assess miR-196a expression in the reference control. For example, in some embodiments that do not involve testing the reference control, the reference control miR-196a expression may be a reference control level of miR-196a expression in cells that are noncancerous that is known in the art, or that has been previously determined by one of ordinary skill in the art, or that has been previously determined by a particular laboratory that will further test the miR-196a expression of the sample. In some specific embodiments, the reference control is non-neoplastic squamous mucosal tissue from esophagus, in which miR-916a ranging from 0.00003 to 0.0005 (mean levels 0.0005±0.0007) or BE, the miR-196a levels ranging from 0.001 to 0.05 (mean levels 0.014±0.015).

In some embodiments, the reference control may or may not include cells. For example, the reference control may an acellular sample of material that has a particular amount of miR-196A that is known to correlate with miR-196a expression in normal or premalignant cells. The reference control, rather than being a sample, may be a particular value of miR-196a expression that has been previously determined.

In some embodiments, the reference control is a single value or range of values of miR-196a expression, and in other embodiments the reference control is a standard curve.

The reference control may be different, depending on the application of the particular method set forth herein. For example, the reference control for diagnosing EA may be different from a reference control for diagnosing dysplasia of the esophagus. For instance, here the reference control could be low grade dysplasia (LGD) (miR-196a levels in LGD ranging from 0.008 to 0.03 (0.013±0.009).

In the context of methods for diagnosing cancer of the esophagus, the reference control miR-196a expression may be miR-196a expression from noncancerous cells. For example, the cells may be normal cells. The cells may be human cells or other mammalian cells. The cells may be of any cell type, but in particular embodiments the cells are mucosal cells. In a specific embodiment, the cells are esophageal mucosal cells. In some embodiments, the reference control miR-196a expression may be miR-196a expression from cells that are premalignant (i.e., dysplasia). The dysplasia may be low grade or high grade.

In the context of methods for diagnosing dysplasia of the esophagus, the reference control miR-196a expression may be miR-196a expression from normal (noncancerous) cells. The cells may be human cells or other mammalian cells. The cells may be of any cell type, but in particular embodiments the cells are mucosal cells. In a specific embodiment, the cells are esophageal mucosal cells.

The methods set forth herein can be applied to determine whether a cell from an esophageal lesion or tumor in a subject is dysplastic or cancerous. Dysplasia may be determined to be low grade or high grade. The subject may be any mammal, such as a mouse, a rat, a rabbit, a dog, a cat, a horse, a cow, a pig, a goat, a primate, or a human. In particular embodiments, the subject is a human. In specific embodiments, the subject is a human that is known or suspected to have a lesion of the esophagus.

In specific embodiments, the subject has a lesion of the esophagus. For example, the lesion of the esophagus may be one for which the diagnosis is unknown, or it may be known or suspected to be a metaplastic lesion of the esophagus or Barrett's esophagus. The lesion may be a lesion confined to the esophageal mucosa, a submucosal lesion, or a lesion that involves both the mucosa and submucosal tissues. Thus, the methods set forth herein may include methods for diagnosing esophageal cancer (such as EA) or a predisposition to the development of adenocarcinoma of the esophagus.

Some embodiments of the present methods further involve obtaining normal cells from the subject with the lesion or from a second subject who does not have a lesion that is known or suspected to be cancerous. In some embodiments, the noncancerous cells are premalignant cells. In particular embodiments, the normal cells are further defined as normal squamous mucosal cells.

Some embodiments of the present methods concern methods for diagnosing a predisposition for cancer or a method for diagnosing a high grade dysplastic lesion. For example, the methods of the present invention may concern a method for identifying a subject with high grade dysplasia of the esophagus. In such an embodiment, for example, the subject with an esophageal lesion may be found to have increased expression of mR-196a in cells of the lesion compared to low grade dysplastic cells or squamous mucosal cells from a subject known to have Barrett's esophagus. Such a subject may thus be one who has high grade dysplasia or EA or is at risk of developing high grade dysplasia or EA. Thus, the methods set forth herein can be applied in diagnosing high grade dysplasia in addition to malignancy.

The subject may be a human who may or may not have symptoms associated with the lesion. For example, where the lesion is an esophageal lesion, the subject may or may not have symptoms of difficulty swallowing or pain from the lesion. The subject may be a subject with no known history of cancer, a subject with a history of cancer that has been previously treated, or a subject with cancer at one site in the body who develops a lesion at a second site of the body.

In certain embodiments, the subject is diagnosed with esophageal cancer if the miR-196a expression in cells of the esophageal lesion is greater than a reference control miR-196a expression that is known to be associated with the expression of miR-196a in dysplastic or premalignant cells. The subject may be one who is diagnosed with esophageal cancer if the miR-196a expression in cells of the esophageal lesion is greater than the reference control.

In some embodiments, the subject is diagnosed with dysplasia of the esophagus if the miR-196a expression in cells of the esophageal lesion is greater than a reference control miR-196a expression that is known to be associated with the expression of miR-196a in normal healthy cells. The clinician may choose to evaluate such a subject particularly closely for progression to EA. For example, repeat testing of miR-196a may be performed to evaluate the subject for further increase in miR-196a, which may subsequently indicate progression to EA. In specific embodiments, the subject has a history of Barrett's esophagus.

In some embodiments, testing cells to assess the level of a miRNA (such as mRNA=196a or a nucleic acid sequence comprising at least 10 contiguous nucleotides of miR-196a) or determining expression of such a miRNA may involve performing any test known to those of ordinary skill in the art. For example, such a test may involve performing fluorescence in situ hybridization (FISH), DNA amplification (such as PCR), microarray hybridization, bead-based hybridization, allele specific oligonucleotide hybridization, size analysis, sequencing, hybridization, 5′ nuclease digestion, allele specific hybridization, primer specific extension, or an oligonucleotide ligation assay.

Some embodiments of the methods set forth herein further involve obtaining a biological sample of a lesion from the subject. In specific embodiments, the method involves obtaining a biological sample of an esophageal tissue of the subject. The method may further involve performing one or more additional diagnostic tests on the subject. For example, the method may further involve performing endoscopy of the esophagus and biopsy of the esophageal lesion by endoscopic guidance.

The methods set forth herein may optionally involve performing one or more additional tests to diagnose cancer. For example, such an additional test may include biopsy or an imaging study (such as CT, PET, or MRI), or performing an additional genetic or laboratory test.

The methods set forth herein may optionally further involve assessing the expression of annexin A1 (ANXA1), keratin 5 (KRT5), small proline-rich protein 2C (SPRR2C), S100 calcium-binding protein A9 (S100A9), cystatin-A (CSTA) or myelin and lymphocyte protein (MAL) in the sample of cells. For example, the method may involve obtaining information regarding whether the expression of ANXA1, KRT5, SPRR2C, S100A9, CSTA or MAL is reduced in the cell sample compared to normal or premalignant cells. Assessing for expression of any of these genes can be by any method known to those of ordinary skill in the art. The method may involve measuring protein function, protein expression, or a combination of both. In some embodiments, mRNA expression is assessed as an indicator of protein expression. Non-limiting examples of detection methods include ELISA immunoassay, radioimmunoassay, chemiluminescence immunoassay, fluorescence immunoassay, cell sorting assay, fluorescence activated cell sorting assay, Western blotting techniques, immunoprecipitation assay, colorimetric or densitometric assay, enzymatic assay, and immunostaining assay.

The present invention also concerns methods for diagnosing cancer or dysplasia in a subject with a lesion that involve determining whether there is reduced expression of annexin A1 (ANXA1), keratin 5 (KRT5), small proline-rich protein 2C (SPRR2C), S100 calcium-binding protein A9 (S100A9), CSTA, and/or MAL in a sample of cells compared to a reference control. In specific embodiments, the cancer is esophageal cancer, breast cancer, or endometrial cancer. The method may further involve determining whether the level of one or more miRNA (such as miR-196a or a nucleic acid comprising 10 or more contiguous nucleotides of SEQ ID NO:1) is increased in cells of the lesion compared to the level of the one or more miRNA in normal or premalignant cells.

The present invention also concerns a method of diagnosing esophageal cancer or a dysplastic lesion of the esophagus, involving: (a) providing a kit that includes at least one PCR primer needed to perform amplification of miR-196a; and (b) distributing said kit to an individual desiring to diagnose esophageal cancer or a dysplastic lesion of the esophagus in a subject. The kit may or may not include one or more additional components. For example, in some embodiments the kit further includes a sealed container comprising a mix of nucleotide triphosphates. In some embodiments the kit further includes a sealed container comprising a polymerase or a reverse transcriptase. The kit may or may not include instructions for use in methods for diagnosing esophageal cancer or diagnosing a dysplastic lesion of the esophagus. In particular embodiments, the kit includes a primer that has SEQ ID NO:11 or SEQ ID NO:12.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device and/or method being employed to determine the value.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The following figures form part of the present specification and are included to further demonstrate certain aspects of the present invention. The invention may be better understood by reference to one or more of these drawings in combination with the detailed description of specific embodiments presented herein.

FIG. 1. Predicted base complementarity of miR-196a to S100A9, SPRR2C, and KRT5. The base complementarity of miR-196a to 3′-UTR binding sites of S100A9, SPRR2C and KRT5 as predicted by Sanger miR-database (http://microrna.sanger.ac.uk/sequences/).

FIG. 2A, 2B, 2C. Micro-RNA 196a levels are characteristically upregulated with progression of esophageal adenocarcinoma. 2A—Real-time q-RT PCR analysis for miR-196a in total RNA isolated from 10 patient samples shows an increase in the levels of miR-196a with neoplastic progression of normal esophageal mucosa (N) to adenocarcinoma (EA). Each data point represents relative expression levels of duplicate reactions in a single experiment. The greatest increase in the levels of miR-196a was observed between N and Barrett's esophagus (BE) with smaller increases in subsequent stages of neoplastic histologic progression from BE to low grade (LGD), LGD to high-grade dysplasia (HGD) and to EA. 2B—Box plots of the relative levels of miR-196a at each stage of progression. Each box shows the variation of relative values of miRs and the black horizontal bar shows the mean value in each box. 2C—Box plots of the log transformed relative values of miR-196a during progression.

FIG. 3A, 3B, 3C, 3D. Inverse correlation of miR-196a levels with mRNA levels of S100A2, SPRR2C and KRT5, three genes characteristically downregulated in EA. 3A—Relative miR-196a levels in EAs from 10 patients as measured by real-time qPCR. The relative levels showed considerable variation among EA, so the EA specimens were arbitrarily grouped into low (samples 1 to 5) and high expressing (6 to 10) tumors. The relative mRNA levels of S100A2 (3B), SPRR2C (3C) and KRT5 (3D) in the same samples were inversely correlated with the relative levels of miR-196a. Each data point represents relative expression levels of duplicate reactions in a single experiment.

FIG. 4A, 4B. 4A—Inverse correlation between miR-196a and its target (CSTA and MAL) mRNA levels in esophageal adenocarcinomas. 4B—Alignment of miR-196a with target sequences.

FIG. 5. Elevation of miR-196a levels in EA cells suppresses expression of S100A9, SPRR2C and KRT5. Elevating the levels of miR-196a by transfection of RNA mimics in the EA cell line BIC1 resulted in suppression of the mRNA levels of all 3 genes, as measured by real-time qPCR assay. The relative mRNA levels of these genes in BIC1 cells transfected with a non-specific miRNA mimic was considered as the respective control.

FIG. 6A, B. SPRR2C, S100A9 and KRT5 mRNAs are direct targets of miR-196a. 6A—The schematic of luciferase assay. To demonstrate the direct targeting of these mRNA by miR-196a, the 3′-UTRs of the genes with the predicted miR-196a binding sites were cloned upstream of the luciferase gene in PGL3 vector plasmid. When transfected into cells the mRNA transcript of the luciferase generated from the vector plasmid has the 3′-UTR of the gene of interest, and the additional miR-196a generated from the RNA mimics binds to the luciferase mRNA and suppresses its levels, resulting in a decrease in luciferase activity. 6B—The plasmid and RNA mimics of miR-196a were cotransfected into BIC1 and OE33 cells and the effect of miR-196a on luciferase gene transcription was measured by luciferase assay. Repression of luciferase activity demonstrated the direct binding and repressive effect of miR-196a on the mRNA of these 3 genes. A non-specific miRNA mimic cotransfected with the luciferase plasmid was used as the respective control. The decrease in the luciferase activity was statistically significant for all targets (p<0.05).

FIG. 7. Sequence complementarity of miRNAs 196a, 196b and 584 to the 3′UTR of ANXA1 mRNA. Nucleotide sequences of miR-196a, -196b and -584, computationally predicted by the Sanger miRNA registry as potential miRNAs targeting ANXA1 mRNA. The base complementarity between the three miRNAs and 3′-UTR of ANXA1 is schematically shown.

FIG. 8A, 8B, 8C, 8D, 8E. Correlation of miR-196a, -196b, and -584 to ANXA1 in esophageal, breast, and endometrial cancer cell lines. (8A) Western blotting analysis of the comparative levels of ANXA1 protein levels in esophageal, breast and endometrial cancer cell lines. Real time q-PCR analysis of ANXA1 mRNA levels (8B) shows inverse correlation with the levels of miR-196a (8C) (correlation coefficient=−0.66, p=0.019) and miR-196b (8D) (correlation coefficient=−0.25, p=0.428) in the 12 cell lines including esophageal (left panels), breast (middle panels) and endometrial cancers (right panels). No negative correlation was observed between ANXA1 mRNA levels and the miR-584 levels (8E) (correlation coefficient=0.228, p=0.475). Note that relative expression levels of microRNA were multiplied by a factor of 1,000 to simplify data representation. The levels of ANXA1 protein corresponded with the observed pattern of ANXA1 mRNA levels in the cell lines.

FIG. 9A, 9B, 9C, 9D. Inverse correlation of miR-196a and ANXA1 in esophageal cancers. Real time q-PCR analysis of the levels of ANXA1 mRNA (9A) and miR-196a (9B) in a set of 10 esophageal tumor samples. Tumors with relatively low levels of ANXA1 mRNA (T1 to T5) showed comparatively high levels of ANXA1 whereas those tumors with relatively higher levels of ANXA1 mRNA (T6 to T10) consistently showed lower levels of miR-196a confirming the inverse correlation (Correlation coefficient=−0.64, p=0.047). (9C) ANXA1 mRNA levels were consistently suppressed in esophageal adenocarcinoma tissue samples in comparison to their paired normal mucosa as seen in a set of 10 paired normal and tumor tissue samples. (9D) A distinct increase of miR-196a levels (10- to 100-fold) was consistently observed in the esophageal tumors in comparison to their respective paired normal mucosa. A distinct inverse correlation observed in each esophageal tissue sample tested reinforced the possibility of miR-196a as a candidate miRNA targeting ANXA1 mRNA.

FIG. 10A, 10B. Transfection with two RNA mimics of miR-196a decreases ANXA1 mRNA and protein levels. (10A) Increasing levels of miR-196a in esophageal (BIC-1, SEG-1), breast (MDA-231) and endometrial (HEC-1B) cancer cell lines by the transfection of two different mimics of miR-196a resulted in a reproducible decrease of ANXA1 mRNA levels as studied by real-time PCR analysis. (10B) Consistent decrease of ANXA1 protein levels was also observed in these cell lines on transfection of the two RNA mimics of miR-196a. The western blotting data for ANXA1 and control β-actin are shown and the quantification of relative levels of ANXA1 protein levels (ANXA1 versus β-actin) under each condition is provided above. ANXA1, annexin A1; qPCR, quantitative PCR.

FIG. 11. RNA mimics of miR-196a directly target 3′-UTR of ANXA1 mRNA. Luciferase assay was conducted in MDA-MB-453 breast cancer cells using the PGL3 luciferase vector, in which a 284-bp section of the 3′-UTR region of the ANXA1 mRNA was cloned downstream of the luciferase gene. A schematic representation of the principle behind the assay employed has been provided (upper panel). Cotransfection of either of the mimics for miR-196a resulted in a drastic suppression of the luciferase activity, indicating the direct targeting of the 3′-UTR of ANXA1 by miR-196a in esophageal (BIC-1 and OE33) and breast cancer cell lines (MDA-453) (lower panels). ANXA1, annexin A1.

FIG. 12A, 12B. Increased levels of miR-196a increased cell proliferation and suppressed apoptosis. (12A) Transfection of both the mimics of miR-196a resulted in a consistently elevated proliferative potential of esophageal, breast and endometrial cancer cell lines within 96 h of transfection, indicating that elevated levels of miR-196a might be involved in neoplastic transformation in cells. The cell proliferation was tested by MTS assay. Results showed are an average of two separate experiments and observed increase in proliferation was statistically significant (P<0.05). (12B) In SEG-1 cells, overexpression of miR-196a suppressed apoptosis induced by staurosporine (100 nM). Reduced extent of cell death was evident after staurosporine treatment in 196a mimic-transfected cells in comparison to the cells transfected with mimic negative control. Cell death was measured by trypan blue dye exclusion assay.

FIG. 13. miR-196a stimulated anchorage-independent colony formation in esophageal cancer cells. BIC-1 and SEG-1 cells were counted and seeded into methylcellulose 24 h after transfection with miR-196a mimics 1 and 2 or mimic negative control. The numbers of colonies were counted after 2 weeks of growth. A distinct increase in the colony-forming ability was observed in both the cell lines transfected with RNA mimics of miR-196a, as evident by the enhanced colony number and size. Colonies were counted under microscope. Colony numbers and representative pictures under each condition are shown.

### DESCRIPTION OF ILLUSTRATIVE EMBODIMENTS

The present invention is based on the finding that certain miRNA, such as miR-196a, are biomarkers for progression of a lesion from a benign to a malignant phenotype. For example, the inventors have found that miR-196a is a biomarker for progression of precancerous lesions of the esophagus to a malignant phenotype. The inventors have also found that miR-196a is involved in the downregulation of certain genes whose expression is characteristically decreased or lost during neoplastic transformation of esophageal tissue. These findings can be applied in the development of methods for the early diagnosis and detection of cancer.

### A. DEFINITIONS

The term “nucleic acid” is well known in the art. A “nucleic acid” as used herein will generally refer to a molecule (i.e., a strand) of DNA or RNA comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. A “gene” refers to coding sequence of a gene product, as well as introns and the promoter of the gene product.

These definitions generally refer to a single-stranded molecule, a double-stranded molecule or a triple-stranded molecule that comprises one or more complementary strand(s) or “complement(s)” of a particular sequence comprising a molecule. As used herein, a single stranded nucleic acid may be denoted by the prefix “ss”, a double stranded nucleic acid by the prefix “ds”, and a triple stranded nucleic acid by the prefix “ts.” In particular aspects, a nucleic acid encodes a protein, polypeptide, or peptide.

As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

As used herein, the term “nucleic acid segment,” are fragments of a nucleic acid, such as a fragment of a microRNA. Various nucleic acid segments may be designed based on a particular nucleic acid sequence, and may be of any length. As used herein, a “probe” generally refers to a nucleic acid used in a detection method or composition. As used herein, a “primer” generally refers to a nucleic acid used in an extension or amplification method or composition.

A nucleic acid is a “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule. In preferred embodiments, a complement is a hybridization probe or amplification primer for the detection of a nucleic acid polymorphism.

As used herein, the term “complementary” or “complement” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. However, in some diagnostic or detection embodiments, completely complementary nucleic acids are preferred.

### B. METHODS OF DETECTING miRNA

Reverse transcription (RT) and the polymerase chain reaction (PCR) are critical to many molecular biology and related applications, particularly gene expression analysis. In these applications, reverse transcription is used to prepare template DNA from an initial RNA sample. The template DNA can then be amplified using PCR to produce a sufficient amount of amplified product for the application of interest. Advances in nucleic acid extraction and amplification have greatly expanded the types of biological samples from which genetic material may be obtained. In particular, PCR has made it possible to obtain sufficient quantities of DNA from fixed tissue samples, archaeological specimens, and quantities of many types of cells that number in the single digits. Detecting, analyzing, and/or quantifying small RNA requires the amplification and detection of RNA with a limited size posing difficulty in analysis of these important RNA targets.

As described herein in more detail, aspects of the methods include detecting one miRNA. In some embodiments, the method comprises the steps of: (a) reverse transcribing one or more RNA target using one or more reverse transcription primer comprising in a 5′ to 3′ direction (i) a primer segment, (ii) a probe segment, and (iii) a 3′ target specific segment that anneals to a RNA target; (b) amplifying one or more RNA or RNA segment from all or part of the reverse transcription reaction using a first amplification primer that anneals to the 3′ end of a reverse transcribed RNA target and a second primer that anneals to a sequence complementary to the primer segment; and (c) detecting amplification of a target nucleic acid.

1. Reverse Transcription Reaction

The components of a reverse transcription reaction may include nuclease free water, reverse transcriptase (RT) buffer, dNTP mix, RT primer, RNase inhibitor, and a reverse transcriptase, are assembled on ice prior to the addition of a RNA template. An example of a RT reaction may include RT buffer, dNTPs, RT primer, an effective amount of a RNase inhibitor(s), an amount of a reverse transcriptase or equivalent enzyme sufficient to produce a DNA template.

The reverse transcription primer typically comprises in a 5′ to 3′ direction (i) a primer segment, (ii) a probe segment, and (iii) a 3′ target specific segment that anneals to an RNA target. The primer can be a unique primer segment. In certain embodiments the primer segment can be a universal primer segment, that is a segment that corresponds to a primer that can be used to prime an RNA target that is a primer that is not specific for a target RNA. The primer segment can be from 5, 6, 7, 8, 9, 10, 11, 12, 13, 14, 15, 16, 17, 18, 19, 20, 21, 22, 23, 24, 25, 26, 27, 28, 29, 30, 40, 50, 100 or more nucleotides in length, including all values and ranges there between.

The probe segment will typically be distinct from a primer segment and/or the target specific segment of the RT primer. The probe segment can be from 5, 6, 7, 8, 9 10, 11, 12, 13, 14, 15, 16, 17, 18, 19, 20. 21. 22. 23. 24. 25. 26, 27, 28, 29, 30, 40, 50, 100 or more nucleotides in length, including all values and ranges there between. In certain aspects the probe will be adjacent to the primer segment, the target specific segment or both the primer segment and the target specific segment.

The 3′ target specific segment comprises a sequence that anneals to a target RNA sequence or its complement and can be from 5, 6, 7, 8, 9, 10, 11, 12, 13, 14, 15, 16, 17, 18, 19, 20, 21, 22, 23, 24, 25, 26, 27, 28, 29, 30, 40, 50, 100 or more nucleotides in length, including all values and ranges there between. The target segment may contain modified bases, such as locked nucleic acid (LNA), 2-O-alkyl, 5′ propyne, G-clamp, or other modified bases. Such bases are typically used to improve binding affinity to the target.

In certain embodiments, target nucleic acid sequences include RNA such as miRNA, siRNA, piwi-interacting RNA, rRNA, tRNA, snRNA, viral RNA and fragments and segments thereof.

A variety of methods are available for obtaining a target nucleic acid sequence. When the nucleic acid target is obtained through isolation from a biological matrix, certain isolation techniques include, but are not limited to, (1) organic extraction followed by ethanol precipitation, e.g., using a phenol/chloroform organic reagent (Ausubel et al., 1993); (2) stationary phase adsorption methods (U.S. Pat. No. 5,234,809; Walsh et al., 1991); and (3) salt-induced nucleic acid precipitation methods (Miller et al., (1988), such precipitation methods being typically referred to as “salting-out” methods. In certain embodiments, the above isolation methods may be preceded by an enzyme digestion step to help eliminate unwanted protein from the sample, e.g., digestion with proteinase K, or other like proteases. See, e.g., U.S. Pat. No. 7,001,724.

In certain embodiments, a RNA may be reverse-transcribed into a DNA target nucleic acid sequence. In certain embodiments, multiple target nucleic acid sequences can be amplified in the same reaction (e.g., in multiplex amplification reactions). Aspects of the invention may include 1, 2, 3, 4, 5, 6, 7, 8, 9, 10 or more different amplifications in a reaction.

In certain aspects, a target nucleic acid sequence is derived from a crude cell lysate. Examples of target nucleic acid sequences include, but are not limited to, nucleic acids from tissue biopsies, buccal swabs, crude bacterial lysates, blood, skin, semen, hair, bone, mucus, saliva, cell cultures, and the like. In still further aspects, target nucleic acid sequences are obtained from a cell, cell line, tissue, or organism that has undergone a treatment, is suspected of contributing or having the propensity of contributing to a pathological condition or is diagnostic of a pathological condition or the risk of developing a pathological condition. In certain embodiments, the methods detect the presence, absence, up-regulation, or down-regulation of certain target nucleic acid sequences in treated cells, cell lines, tissues, or organisms.

In yet further aspects, a target nucleic acid sequence(s) is obtained from a single cell, tens of cells, hundreds of cells or more. In some aspects, a target nucleic acid sequence is extracted from cells of a single organism. A target nucleic acid sequence concentration in a PCR reaction may range from about 1, 100, 1,000 to about 100,000, 1,000,000, 10,000,000 molecules per reaction, including all values there between.

Certain embodiments of the invention are directed to detection and quantitation of miRNA. The miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved in animals by a ribonuclease III-like nuclease enzyme called Dicer. The processed miRNA is typically a portion of the stem.

The processed miRNA (also referred to as “mature miRNA”) become part of a large complex to down-regulate a particular target gene. Examples of animal miRNAs include those that imperfectly basepair with the target, which halts translation (Olsen et al., 1999; Seggerson et al., 2002).

miRNAs can be employed in diagnostic, therapeutic, or prognostic applications, particularly those related to pathological conditions described herein. They may be isolated and/or purified. The term “miRNA,” includes the processed RNA and its precursor.

Target RNA may be at least, at most, or about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides or kilobases, or any range derivable therein, in length. In many embodiments, miRNA are 19-24 nucleotides in length depending on the length of the processed miRNA and any flanking regions added. miRNA precursors are generally between 62 and 110 nucleotides in humans.

It is understood that a miRNA is derived from genomic sequences or a gene. In this respect, the term “gene” is used for simplicity to refer to the genomic sequence encoding the precursor miRNA for a given miRNA. However, embodiments of the invention may involve genomic sequences of a miRNA that are involved in its expression, such as a promoter or other regulatory sequences.

As used herein, the term “reverse transcriptase (RT)” is used in its broadest sense to refer to any enzyme that exhibits reverse transcription activity as measured by methods known in the art. Reverse transcriptase activity refers to the ability of an enzyme to synthesize a DNA strand utilizing an RNA strand as a template. A “reverse transcriptase” of the present invention, therefore, includes reverse transcriptases from retroviruses, other viruses, and bacteria, as well as a DNA polymerase exhibiting reverse transcriptase activity, such as Tth DNA polymerase, Taq DNA polymerase, Tne DNA polymerase, Tma DNA polymerase, etc.

2. Amplification Reactions

A typical polymerase chain reaction (PCR) includes three steps: a denaturing step in which a target nucleic acid is denatured; an annealing step in which a set of PCR primers (forward and reverse (backward) primers) anneal to complementary DNA strands; and an elongation step in which a thermostable DNA polymerase elongates the primers. By repeating this step multiple times, a DNA fragment is amplified to produce an amplicon, corresponding to the target DNA sequence. Typical PCR reactions include 30 or more cycles of denaturation, annealing and elongation. In many cases, the annealing and elongation steps can be performed concurrently, in which case the cycle contains only two steps.

Suitable amplification methods include, but are not limited to PCR (Innis et al., 1990), ligase chain reaction (LCR) (see Wu and Wallace, 1989; Landegren et al., 1988 and Barringer et al., 1990), transcription amplification (Kwoh et al., 1989), and self-sustained sequence replication (Guatelli et al. 1990).

In certain aspects, each primer is sufficiently long to prime the template-directed synthesis of the target nucleic acid sequence under the conditions of the amplification reaction. In certain embodiments, the lengths of the primers depends on many factors, including, but not limited to, the desired hybridization temperature between the primers, the target nucleic acid sequence and the complexity of the different target nucleic acid sequences to be amplified, and other factors. In certain embodiments, a primer is about 15 to about 35 nucleotides in length. In certain embodiments, a primer is fewer than 15 nucleotides in length. In certain embodiments, a primer is greater than 35 nucleotides in length.

In certain embodiments, a probe may include Watson-Crick bases or modified bases. Modified bases include, but are not limited to, the AEGIS bases (from Eragen Biosciences), which have been described, e.g., in U.S. Pat. Nos. 5,432,272; 5,965,364; and 6,001,983. In certain aspects, bases are joined by a natural phosphodiester bond or a different chemical linkage. Different chemical linkages include, but are not limited to, a peptide bond or an LNA linkage, which is described, e.g., in published PCT applications WO 00/56748 and WO 00/66604.

In a further aspect, oligonucleotide probes present in a multiplex amplification are suitable for monitoring the amount of amplification product produced as a function of time. Such oligonucleotide probes include, but are not limited to, the 5′-exonuclease assay (e.g., TaqMan™) probes (see above and also U.S. Pat. No. 5,538,848), and others well-known to those of ordinary skill in the art. In certain aspects, a label is attached to one or more probes and has one or more of the following properties: (i) provides a detectable signal; (ii) interacts with a second label to modify the detectable signal provided by the second label, e.g., FRET (Fluorescent Resonance Energy Transfer); (iii) stabilizes hybridization, e.g., duplex formation; and (iv) provides a member of a binding complex or affinity set, e.g., affinity, antibody/antigen, ionic complexes, hapten/ligand (e.g., biotin/avidin).

A polymerase is an enzyme that is capable of catalyzing polymerization of nucleic acids such as RNA and DNA. Numerous diagnostic and scientific applications use polymerases to amplify or synthesize polynucleotides from nucleic acid templates. One application of this method is detecting or isolating nucleic acids present in low copy numbers. In certain aspects, a polymerase is active at 37, 42, 50, 60, 70, 80, 90 degrees C. or higher. In some aspects the polymerase is a thermostable polymerase. Exemplary thermostable polymerases include, but are not limited to, Thermus thermophilus HB8 (see, e.g., U.S. Pat. No. 5,789,224 and U.S. publication 20030194726); mutant Thermus oshimai; Thermus scotoductus; Thermus thermophilus 1B21; Thermus thermophilus GK24; Thermus aquaticus polymerase (AmpliTaq® FS or Taq (G46D; F667Y) (see e.g., U.S. Pat. No. 5,614,365), Taq (G46D; F667Y; E6811), and Taq (G46D; F667Y; T664N; R660G); Pyrococcus furiosus polymerase; Thermococcus gorgonarius polymerase; Pyrococcus species GB-D polymerase; Thermococcus sp. (strain 9.degree. N-7) polymerase; Bacillus stearothermophilus polymerase; Tsp polymerase; ThermalAce™ polymerase (Invitrogen); Thermus flavus polymerase; Thermus litoralis polymerase and mutants or variants thereof.

### C. SAMPLE PREPARATION

The methods of the invention are not limited to any particular method of sample preparation. A large number of well-known methods are suitable for this invention.

In one embodiment, such a sample is a homogenate of cells or tissues or other biological samples. In certain aspects, such sample is a total RNA preparation of a biological sample. In a further aspect, such a sample may be a small RNA preparation of a biological sample.

Biological samples may be of any biological tissue or fluid or cells. Frequently the sample will be a “clinical sample” which is a sample derived from a patient. Clinical samples provide a rich source of information regarding gene expression, a pathological or pre-pathological condition, and/or a diagnostic parameter. Some embodiments of the invention are employed to detect mutations and to identify the function of mutations. Such embodiments have extensive applications in clinical diagnostics and clinical studies. Typical clinical samples include, but are not limited to, tissue or fine needle biopsy samples. Biological samples may also include sections of tissues such as frozen sections, or sections otherwise preserved or mounted for sectioning and/or histological analysis. In certain aspects, samples are fresh samples or fixed samples, such as formalin or formaldehyde fixed paraffin embedded samples (FFPE).

One of skill in the art would appreciate that it is desirable to inhibit or destroy RNase present in samples before the samples can be analyzed. Methods of inhibiting or destroying nucleases are well known in the art. In some aspects, cells or tissues are homogenized in the presence of chaotropic agents to inhibit nucleases. In some other aspects, RNase are inhibited or destroyed by heat treatment followed by proteinase treatment.

Methods of isolating RNA are also well known to those of skill in the art. For example, methods of isolation and purification of nucleic acids are described in detail in Chapter 3 of Laboratory Techniques in Biochemistry and Molecular Biology: Hybridization With Nucleic Acid Probes (1993). In a certain aspects, the RNA is isolated from a given sample using, for example, an acid guanidinium-phenol-chloroform extraction method (see, e.g., Sambrook et al., (1989), or Ausubel et al. (1987). In one aspect, total RNA can be isolated from mammalian cells using RNeasy™ Total RNA isolation kit (QIAGEN). If mammalian tissue is used as the source of RNA, a commercial reagent such as TRIzol™ Reagent (GIBCOL Life Technologies) may be used. A second cleanup after the ethanol precipitation step in the TRIzol™ extraction using Rneasy™ total RNA isolation kit may be beneficial. Hot phenol protocol described by Schmitt et al., (1990) is useful for isolating total RNA for yeast cells.

### D. PROTEINS

Certain embodiments of the present invention concern determining expression of particular proteins as set forth herein. The full-length amino acid sequence of the human keratin 5 (KRT5) protein is provided herein, and is designated SEQ ID NO:2. The full-length amino acid sequence of the human small proline-rich protein 2C (SPRR2C) protein is provided herein, and is designated SEQ ID NO:3. The full-length amino acid sequence of the human S100 calcium-binding protein A9 (S100A9) is provided herein, and is designated SEQ ID NO:4.

Any method known to those of skill in the art can be used to quantify functional activity of a protein. For example, protein expression can be measured by western blot analysis, immunohistochemistry, protein array, or any method known to those of skill in the art. Alternatively, protein expression can be measured indirectly, such as by measurement of mRNA transcription and/or stability, measurement of gene copy number of the gene in a cell, or any method known to those of skill in the art.

### E. DIAGNOSTICS

Embodiments of the invention include methods for diagnosing and/or assessing a condition or potential condition in a patient comprising measuring expression of one or more RNA, such as a miRNA, in a sample from a patient. The difference in the expression in the sample from a patient and a reference, such as expression in a normal or non-pathologic sample, is indicative of a pathologic, disease, or cancerous condition, or risk thereof. A sample may be taken from a patient having or suspected of having a disease or pathological condition. In certain aspects, the sample can be, but is not limited to tissue (e.g., biopsy, particularly fine needle biopsy), blood, serum, plasma, or a pancreatic juice samples. The sample can be fresh, frozen, fixed (e.g., formalin fixed), or embedded (e.g., paraffin embedded).

The present invention is of particular interest in the diagnostic screening of RNA samples for many diseases or conditions. In certain embodiments, diagnostic methods involve identifying one or more RNA, such as miRNAs or mRNAs, differentially expressed in a sample that are indicative of a disease or condition (non-normal sample). In certain embodiments, diagnosing a disease or condition involves detecting and/or quantifying an expressed miRNA. RNAs clearly linked to a disease phenotype are referred to as “biomarkers.”

Particularly the methods can be used to evaluate samples with respect to diseases or conditions that include, but are not limited to esophageal cancer.

It is specifically contemplated that the invention can be used to evaluate differences between stages of disease, such as between hyperplasia, neoplasia, pre-cancer and cancer, or between a primary tumor and a metastasized tumor.

### F. PERSONAL HISTORY MEASURES AND TESTS FOR DIAGNOSIS OF ESOPHAGEAL CANCER

In addition to use of the genetic analysis disclosed herein, the present invention makes use of additional factors in gauging an individual's risk for developing esophageal cancer. Other testing procedures for the diagnosis of esophageal cancer may be employed. Examples of such tests include an endoscopic examination of the esophagus (esophagoscopy) or an imaging study. Esophagoscopy involves the insertion of a thin, flexible, lighted tube into the esophagus. A camera in the endoscope allows the physician to view the esophagus, and to examine it for abnormalities. A tissue sample (biopsy) can be collected for microscopic examination or for practice of a method of the present invention, or for microscopic analysis to assess for cancerous cells. A barium X-ray uses a contrast fluid to produce an image of the lining of the esophagus. This image reveals the presence of any strictures or bumps in the esophagus wall. Other tests include endoscopic ultrasound, PET scans, bronchoscopy, thoracoscopy and laparoscopy.

The methods set forth herein may involve determining whether the subject has other risk factors for the development of cancer of the esophagus. These other risk factors include increasing age, gender (more common in males than in females), history of gastroesophageal reflux disease, history of tobacco use, family history of cancer, previous history of cancer, history of alcohol intake, obesity, history of diet low in fruits and vegetables, history of drinking of very hot liquids, occupational exposure to solvents or toxins, lye ingestion, achalasia, tylosis, and esophageal webs.

### G. KITS

The present invention also contemplates the preparation of kits for use in accordance with the present invention. Suitable kits include various reagents for use in accordance with the present invention in suitable containers and packaging materials, including tubes, vials, and shrink-wrapped and blow-molded packages.

Materials suitable for inclusion in a kit in accordance with the present invention comprises one or more of the following:
gene specific PCR primer pairs (oligonucleotides) that anneal to sequences that flank the DNA encoding the miRNA of interest;
reagents capable of amplifying a specific sequence domain in either genomic DNA or cDNA without the requirement of performing PCR;
reagents such as agarose or polyacrylamide and a buffer to be used in electrophoresis, HPLC columns, SSCP gels, formamide gels or a matrix support;
reagents used in bead-based (luminex) gene expression detection methods.

In a non-limiting example, reagents for reverse transcribing a miRNA, using a RT primer comprising in a 5″ to 3′ direction a primer segment, a probe segment, and a target specific annealing segment are included in a kit. The kit may also include one or more primers to sites on an RNA. Such a kit may include one or more buffers, such as a reaction, amplification, and/or a transcription buffer, and components for isolating and/or detecting an amplification product, such as probe or label.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in one or more vial.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power.

The container means will generally include at least one vial, test tube, flask, bottle, syringe and/or other container means, into which reactions are placed or allocated and/or reaction methods are performed. The kits may also comprise a second container means for containing a buffer and/or other diluent.

A kit may also include instructions for employing the kit components as well the use of any other reagent not included in the kit. Instructions may include variations that can be implemented.

The kits of the invention are not limited to the particular items identified above and may include any labeling reagent or reagent that promotes or facilitates the labeling of a nucleic acid.

### H. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### MicroRNA-196a is a Potential Marker of Progression During Barrett's Metaplasia-Dysplasia-Invasive Adenocarcinoma Sequence in Esophagus

### Materials and Methods

Clinical and pathologic characteristics and patient samples in progression study. The institutional database of the Department of Pathology at The University of Texas M. D. Anderson Cancer Center was searched and 10 patients who were diagnosed with early EA who underwent esophagogastrectomy without preoperative neoadjuvant therapy were selected. A detailed retrospective chart review was performed to document stage, EGD findings, and treatment. The study was approved by the institutional review board with waiver of informed consent.

Endoscopic ultrasonography, EGD, computed tomography (CT) and positron emission tomography (PET) scan findings were reviewed for location and stage of tumor, and the presence and extent of BE. All patients were retrospectively clinically staged as per the sixth edition of the American Joint Commission on Cancer staging system (Springer, 2006).

Hematoxylin and eosin (H&E) stained slides from multiple preoperative biopsies and esophagogastrectomy specimens were reviewed for presence of BE, degree of dysplasia, and histologic subtype of carcinoma. In addition, the depth of invasion and regional lymph node status were evaluated in 10 resection specimens for early EA with mucosal or submucosal invasion. The resection specimens were reviewed by two gastrointestinal pathologists to identify the foci of non-neoplastic squamous mucosa (NSM), Barrett's esophagus (BE), low grade dysplasia (LGD), high-grade dysplasia (HGD) and invasive adenocarcinoma. The slides of NSM, BE, LGD, HGD, and adenocarcinoma were selected for micro-dissection. In all 10 patients, adequate material was available from NSM, BE, LGD and HGD. In addition, the adenocarcinoma component was available in five of these 10 patients. Eight slides with 5-micron thick sections were prepared from the formalin fixed paraffin block without trimming the block for each lesion from the surgical resection specimens. These slides were H&E stained and the lesion of interest was manually micro-dissected.

Identification of miR-196a targets and clinical and pathologic characteristics of patient samples for miR-196a and its target correlative study. From the list of genes down regulated during BE progression to EA in the study of Kimchi et al. (Kimchi et al. 2005), 13 genes (Table 1) were identified as potential targets of miR-196a on the basis of the sequence complementarity between their 3′-untranslated regions (3′-UTR) and miR-196a using Sanger microRNA database (http://microrna.sanger.ac.uk/sequences/). Interestingly, we noted that seven of the 13 miR-196a in silico targets were also downregulated in tumors resistant to preoperative chemoradiation (Table 1).

TABLE 1
Potential targets of miR-196a downregulated during progression
of BE-EA (Kimchi et al., 2005; Selaru et al., 2002).

| Gene Symbol | Gene Name |
|---|---|
| ANXA1* | Annexin A1 |
| S100A9* | S100 calcium binding protein A9 |
| SPRR2C* | Small proline-rich protein 2C |
| KRT5* | Keratin 5 |
| CLCA2* | CLCA family member 2, |
| | chloride channel regulator |
| CYP4B1 | Cytochrome P450, family 4, |
| | subfamily B, polypeptide 1 |
| KRT4* | Keratin 4 |
| LDOC1 | Leucine zipper, downregulated |
| | in cancer 1 |
| LTA4H | Leukotriene A4 hydrolase |
| PTN* | Pleiotrophin |
| MAL | Mal, T-cell differentiation |
| | protein |
| TPD52L1 | Tumor protein D52-like 1 |
| VSNL1 | Visinin-like 1 |

*Genes downregulated in chemoradiation-resistant tumors (Luthra et al., 2007; Luthra et al., 2006).

Table 1 lists genes that are computationally predicted to be potential targets of miR-196a and whose expression is known to be altered during the neoplastic progression of normal esophageal mucosa to adenocarcinoma. Genes which are also known to be downregulated in tumors resistant to chemoradiation are marked with “*” (Luthra et al., 2006). Interestingly, it was noted that seven of the 13 miR-196a in silico targets were also downregulated in tumors resistant to preoperative chemoradiation (Table 1) (Luthra et al., 2007; Luthra et al., 2006). To determine whether increased miR-196a levels correlate with decreased expression levels of computationally predicted targets, S100A9, SPRR2C and KRT5 mRNA levels were measured in EA specimens from 10 additional patients. The selection of the target genes was based on their downregulation during BE progression to EA (Kimchi et al., 2005) and in EA (Luthra et al., 2007; Luthra et al., 2006; Sato et al., 2006).

Because the specimens used in the current progression studies were from early cancers, it was not possible to harvest fresh tumor tissue for miR-196a and mRNA due to inability to identify invasive carcinoma by gross examination. Thus, specimens for target correlative study included fresh frozen and histologically confirmed adenocarcinomas from patients who had advanced loco-regional disease. Five of the 10 patients for the target correlative study were included in our prior study (Luthra et al., 2007).

Real-time quantitative polymerase chain reaction (real-time qPCR) for miRNA expression analysis. Total RNA from microdissected tissue was isolated by using RecoverALL™ Total Nucleic Acid isolation kit (Ambion/Applied Biosciences Austin, Tex.) according to manufacturer's instructions. The RNA yield was measured using Nanodrop (Thermo Scientific, Wilmington, Del.).

The levels of miR-196a and miR-16 were determined by stem loop real-time qPCR using gene-specific TaqMan® minor groove binding (MGB) primers according to the TaqMan MicroRNA Assay protocol (PE Applied Biosystems, Foster City, Calif.). The reverse transcription (RT) reaction was performed with 50 ng of total RNA from each specimen in a total volume of 7.5 μl using 50 nM gene-specific stem-loop primer, 1×RT buffer, 0.25 mM each of d NTPs, 3.33 U/μl MultiScribe reverse transcriptase, and 0.25 U/μl RNase inhibitor (PE Applied Biosystems). The reaction mix was incubated in an Applied Biosystem's 9800 ThermoCycler in a 96-well plate for 30 min at 16° C., 30 min at 42° C., and 5 min at 85° C. and then held at 4° C. Each miRNA was amplified individually and in duplicates.

Real-time qPCR was performed using an Applied Biosystems 7900 Sequence Detection system in 10 μl volume that included 0.67 ml RT product, 1× TaqMan Universal PCR master mix and 1 μl of gene-specific primers and probe mix from the TaqMan microRNA assay. The PCR thermal cycling conditions were as follows: 10 min at 95° C. for AmpliTaq Gold activation and 40 cycles for the melting (95° C., 15s) and annealing/extension (60° C. for 1 min) steps. Each miRNA was amplified individually and in duplicate. Default threshold settings were used to determine threshold cycle (CT).

Comparative CT method (2^{-ΔCT}) for relative quantification of miRNA expression. miR-16 was used as normalizer because this miRNA showed minimal variation in esophageal, breast and endometrial cell lines and also in a pilot miR profiling studies of esophageal cancers. The relative expression levels of each miRNA in comparison to the normalizer were then calculated using the formula 2^{-ΔCT} where ΔCT represents the difference between each target gene and the normalizer (average CT for the target minus average CT for miR-16).

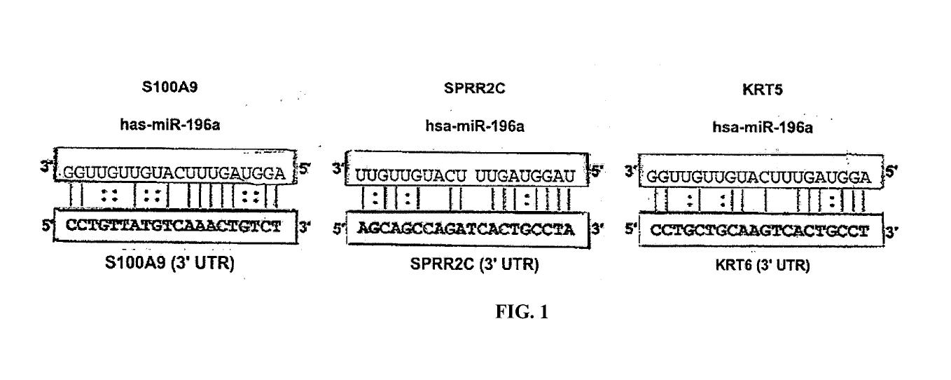

Quantitation of SPRR2C, S100A9, and KRT5 mRNA levels in a EA cell line transfected with miR-196a mimic. The base complementarity of miR-196a with 3′-untranslated region (3′-UTR) of SPRR2C, S100A9 and KRT5 is shown in FIG. 1. To evaluate if overexpression of miR-196a resulted in reduction of these potential target mRNA levels, the EA cell line BIC-1 was transfected with a RNA mimic of miR-196a purchased from Dharmacon, Inc. (Chicago, Ill.). The miRNA mimic is a synthetic double-stranded RNA oligonucleotide, which on delivery generates higher levels of the respective miRNA in the cells, thus amplifying its effects. The mimic was transfected into the cultured cells using DharmaFECT Duo transfection reagent (Dharmacon, Inc.) at a final concentration of 40 nM. After 48 hrs, total RNA was extracted from the cells and the levels of miR-196a and SPRR2C, S100A9, and KRT5 mRNA were measured by real-time qPCR analysis, as described above. A non-specific miRNA mimic (Dharmacon, Inc.) was used as an appropriate negative control.

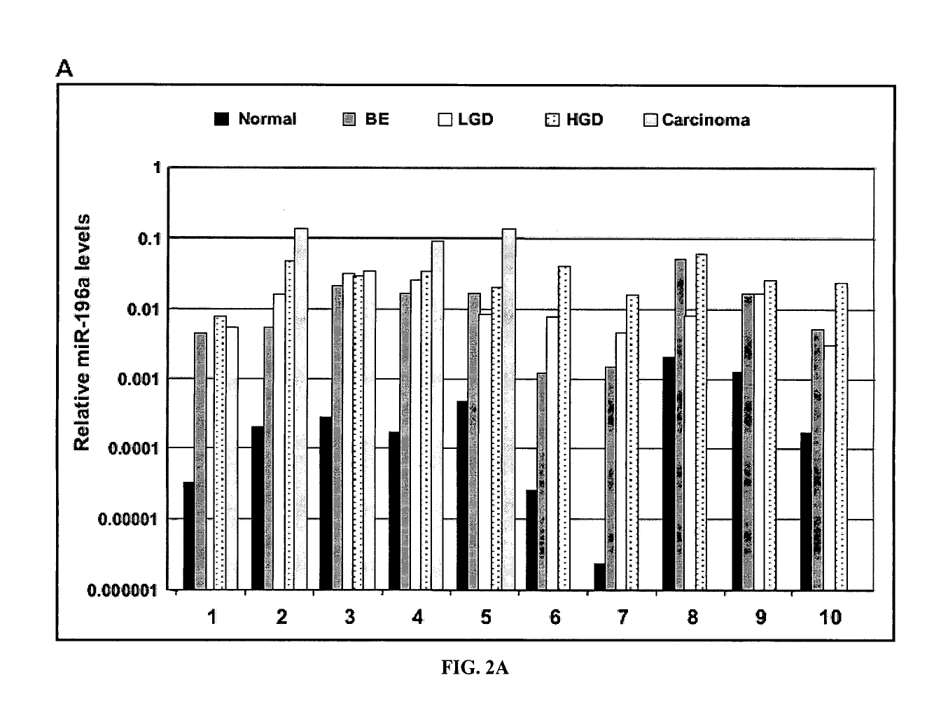

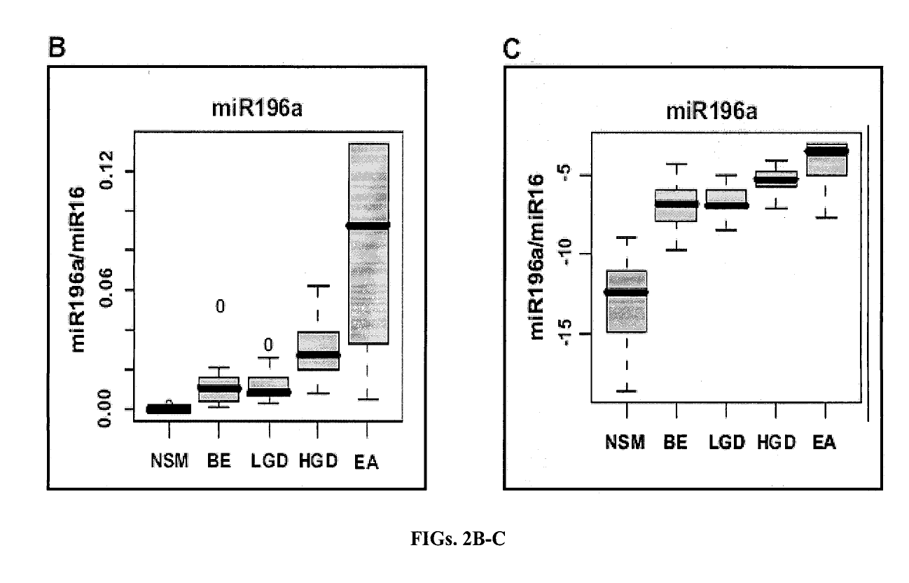

Generation of the target 3′-untranslated region (3′-UTR) luciferase reporter constructs. To further confirm that KRT5, SPR2C and S100A9 are true in vitro target of miR-196a, regions of 3′-UTR of these genes containing the putative miR-196a recognition site (FIG. 2) were amplified using genomic DNA and then cloned into the pGL3 control vector (Promega Corporation, Madison, Wis.) at the XbaI site (Schematic in FIG. 2) and confirmed by sequencing. The size of the fragment amplified and the primers used for amplification are shown in Table 2. Shown in Table 2 is the size of the 3′-UTR region of the three target genes with the potential binding region of miR-196a and the primers used to amplify these regions from the genomic DNA.

TABLE 2
Primers used to amplify the 3′-UTR of
the miR-196a target genes

| Gene | Size | Primers | |
|---|---|---|---|
| A. S100A9 | 365 bp | F: | 5′-tagTCTAGAggtcatagaacacatc-3′ |
| | | | (SEQ ID NO: 5) |
| | | R: | 5′-tagTCTAGAgacttggaggaagagac-3′ |
| | | | (SEQ ID NO: 6) |
| B. SPRR2C | 365 bp | F: | 5′-tagTCTAGAcagcttcggaattcatc-3′ |
| | | | (SEQ ID NO: 7) |
| | | R: | 5′-tagTCTAGAgctactttattcagggag-3′ |
| | | | (SEQ ID NO: 8) |
| C. KRT5 | 373 bp | F: | 5′-tagACTAGTgaacctgctgcaagt-3′ |
| | | | (SEQ ID NO: 9) |
| | | R: | 5′-tagACTAGTatattataaaagcat-3′ |
| | | | (SEQ ID NO: 10) |

Cotransfection of the plasmid and the RNA mimics into the cells was accomplished using DharmaFECT Duo transfection reagent (Dharmacon, Inc.). Luciferase assay was performed 48 h after transfection using the Dual Luciferase Reporter Assay System kit (Promega Corporation). A non-specific miRNA mimic was used as an appropriate negative control.

Quantitative analysis of SPRR2C, S100A9, and KRT5 mRNA levels in tumors. For cDNA synthesis, 200 ng total RNA from each adenocarcinoma sample was reverse transcribed in a final volume of 20 μl using random primers and SuperScript II™ Reverse Transcriptase (Invitrogen, Carlsbad, Calif.). The TaqMan minor groove binder probe and the ABI Prism® 7900 HT Sequence Detection System (PE Applied Biosystems) were used to perform real-time qPCR. The primers and probes for SPRR2C, S100A9, KRT5 and an internal control, glucuronidase beta (GUSB), were obtained from PE Applied Biosystems via their Assays-on-Demand gene expression products services. PCR assays included 10 μl of TaqMan Universal Master Mix No Amperase UNG (2×), 1 μl of 20× Assays-on-Demand Gene Expression Assay Mix, and 2 μl of cDNA diluted in RNase-free water in a final volume of 20 μl. ABI Prism® 7900 HT Sequence Detection System and cycling conditions identical to those described above for miRNA were used for mRNA expression analysis.

Each target was amplified individually and in duplicate. The relative levels of each target were then calculated using average of the duplicates on the basis of the difference between amplification of the target and GUSB mRNA using the ΔCT method as described above.

The qPCR data for both miR-196a as well as its targets were averages of duplicate reactions in a single experiment.

Statistical methods. One-way within-subjects analysis of variance (ANOVA) was used to test against the null hypothesis that there is no overall difference in relative expression levels among normal, precancerous and cancerous tissue (Chambers et al., 1992). For statistically significant ANOVA results, student's t-tests were performed to further test the difference of miRNA expression between any two neoplastic progression stages. Holm's method was applied to adjust p-values of t-tests to correct multiple comparisons (Holm, 1979). Relative expression levels were log transformed before analysis. p-values ≦0.05 were considered statistically significant.

Spearman's rank correlation coefficient was used to measure the rank-based association between miR-196a levels and SPRR2C, S100A9, and KRT5 mRNA levels among the 10 cancer specimens. To assess the significance of coefficients, the p-values were computed using algorithm AS 89 (Best et al., 1975).

### Results

The study population for miRNA analysis in progression specimens comprised men with an average age of 65 yrs (range 57-74 yrs). All patients had long-segment BE on EGD and resection specimens. Six cases had adenocarcinoma with submucosal invasion and 4 cases had intramucosal adenocarcinoma. Seven tumors were moderately differentiated adenocarcinoma, 2 tumors were well-differentiated adenocarcinoma and 1 tumor was poorly differentiated adenocarcinoma. The patient population for the correlative study of miR-196a and its targets included 9 men and 1 woman with an average age of 62 yrs (range 40-79 yrs). All patients had advanced loco-regional disease (stage II or III) on pretreatment staging. Nine tumors were moderately differentiated and one tumor was poorly differentiated. No tumor in either population had signet ring cell histology on pretreatment biopsies.

miR-196a levels during progression of BE-dysplasia-EA. The relative expression levels of miR-196a in each histologic type of lesion at different stages of progression in 10 patients are shown in FIG. 2A. miR-196a levels increased incrementally with each stage of progression from normal mucosa to EA. The box plot of relative levels of miR-196a for the samples analyzed (FIG. 2B, 2C) reflects the progressive increase in miR-196a levels and illustrates that the progression of NSM-BE-LGD-HGD-EA was associated with a concomitant increase of miR-196a levels. The mean miR-196a levels were 0.0005±0.0007 in NSM, 0.014±0.015 in BE, 0.013±0.009 in LGD, 0.03±0.016 HGD and 0.079±0.058 in EA. The differences among different stages were statistically significant by a one-way within-subjects ANOVA with p<0.0001. In patients 1-5 with available EA, a 10-100 fold increase in miR-196a levels was evident in EA when compared with the normal as reflected by pair wise comparisons (p=0.0005, student's t-test with p-values adjusted by the Holm's method). The pair wise comparisons also indicate that the miR-196a levels in each precancerous lesion were significantly higher than control squamous mucosa: NSM versus BE (p=0.00001), versus LGD (p=0.001), versus HGD (p=0.00006); and BE versus HGD (p=0.045); and LGD versus HGD (p=0.02).

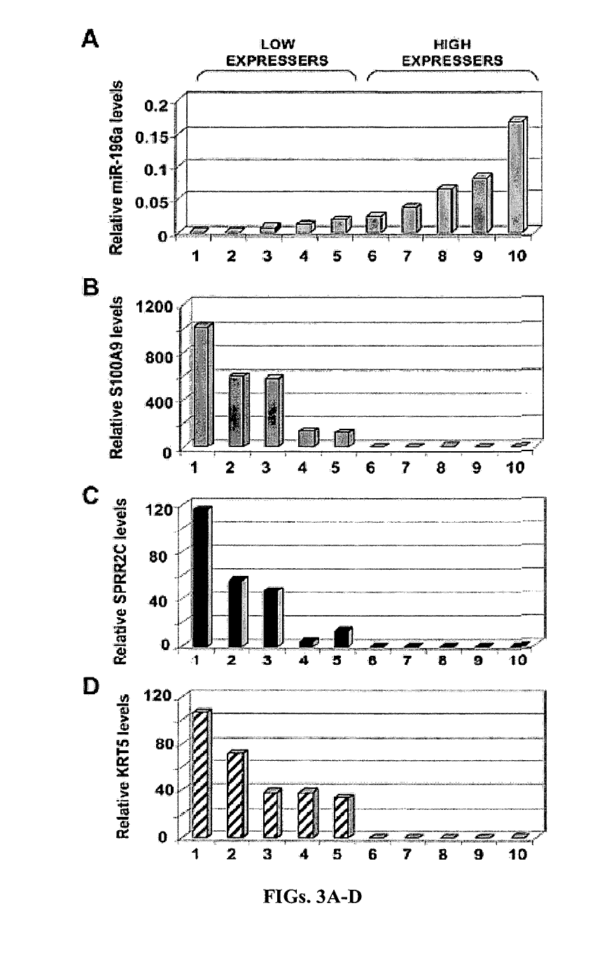

Correlation of miR-196a levels with mRNA levels of predicted targets, SPRR2C, S100A9, KRT5, CSTA and MAL in EA. To test whether down regulation of SPRR2C, S100A9 and KRT5 genes during the progression of BE-EA (Kimchi et al., 2005) is due to increased miR-196a levels, the correlation between their mRNA levels to mR-196a levels in EA specimens from 10 additional patients was tested. miR-196a levels varied considerably in tumors specimens, ranging from 0.0009 to 1.69 and the specimens were arbitrarily separated into two categories expressing relatively low (samples 1 to 5) and high (samples 6 to 10) levels of miR-196a (FIG. 3A). Although, the mean miR-196a expression was comparatively low in the low expressing category of tumors (0.0081±0.0085), this level was substantially higher in comparison to the mean miR-196a levels in the normal mucosa (0.00047±0.00067).

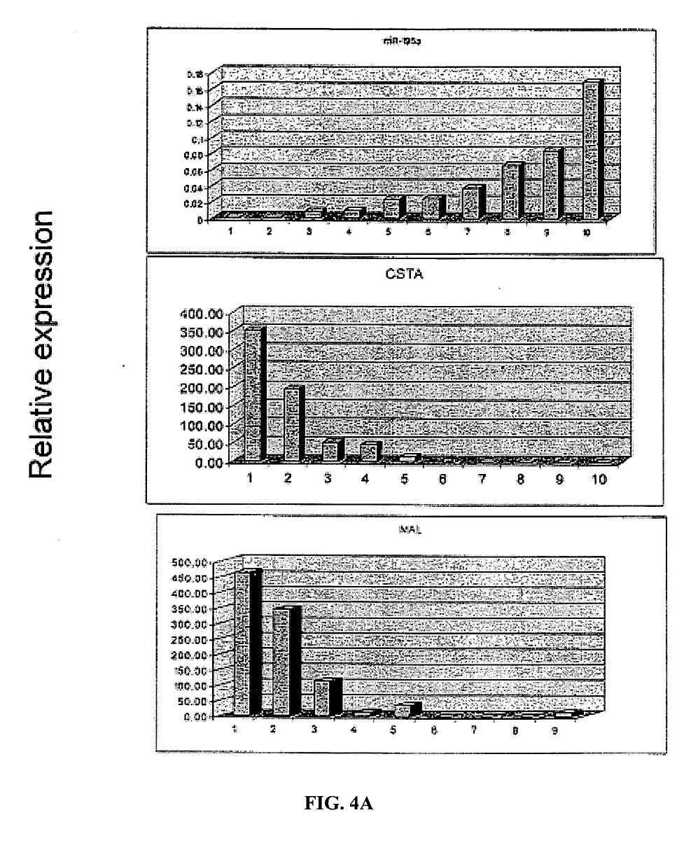

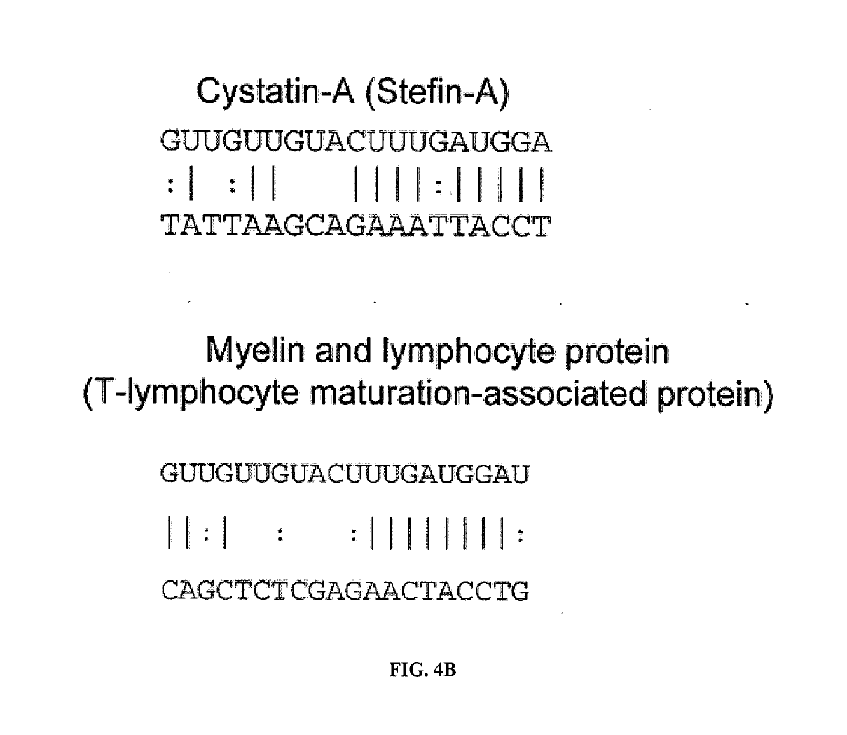

The mRNA levels of SPRR2C, S100A9 and KRT5 also varied significantly among the tumors with levels ranging from 0.0 to 115.3 (23.46±38.36), from 0.15 to 1021.87 (246.4±359.3), from 0.0 to 106.68 (29.0±36.87), respectively (FIG. 3B-D). The tumor specimens with low miR-196a expression showed high mRNA levels of SPRR2C, S100A9 and KRT5. Conversely, specimens with high miR-196a levels showed low expression of its targets. For instance, the mean mRNA levels of SPRR2C, S100A9 and KRT5 were 47±44 (range, 3.78 to 115.3), 491.4±374.77 (range, 123.6 to 1021.9) and 57.9±31.2 (range, 33.63 to 106.68) respectively in low expressers of miR-196a. On the other hand, in the miR-196a high-expressing category of tumors, the mean mRNA levels of SPRR2C, S100A9 and KRT5 were 0.003±0.005, 1.47±1.78 and 0.16±0.35 respectively. The mRNA levels of the targets correlated inversely with miR-196a levels (Spearman's rank correlation coefficients of −0.77, −0.81 and −0.78 for SPRR2C, S100A9 and KRT5 respectively, p<0.01). Similar inverse correlation is also observed between miR-196a and additional targets CSTA and MAL (FIGS. 4A, 4 B)

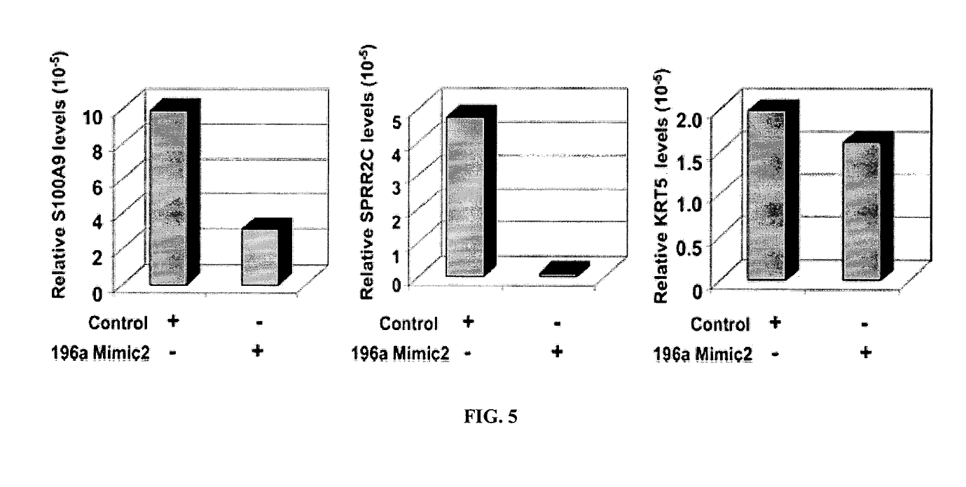

miR-196a mimic suppresses SPRR2C, S100A9 and KRT5 mRNA levels in esophageal cancer cell line. The inverse correlation observed between the levels of miR-196a and mRNAs of the three genes suggests that these mRNAs are likely targets of mR-196a. The sequence complementarity of miR-196a and computationally identified 3′-UTR binding sites of these three mRNAs is shown in FIG. 1. To further confirm that these mRNAs are indeed in vitro cellular targets, the effect of increasing the levels of miR-196a on the mRNA levels of these genes in a esophageal cancer cell line was tested. This was achieved by the transfection of a RNA mimic of miR-196a into BIC1 cell line derived from EA. The miR-196a miRNA mimic was a synthetic RNA oligonucleotide that on delivery generated higher levels of miR-196a in the cells. Increasing miR-196a levels in BIC-1 cells for 48 hrs with the mimic resulted in 69%, 98% and 20% decrease in S100A9, SPRR2C and KRT5 mRNA levels respectively, compared with the respective controls wherein the cells were transfected with a non-specific negative control RNA mimic (FIG. 5).

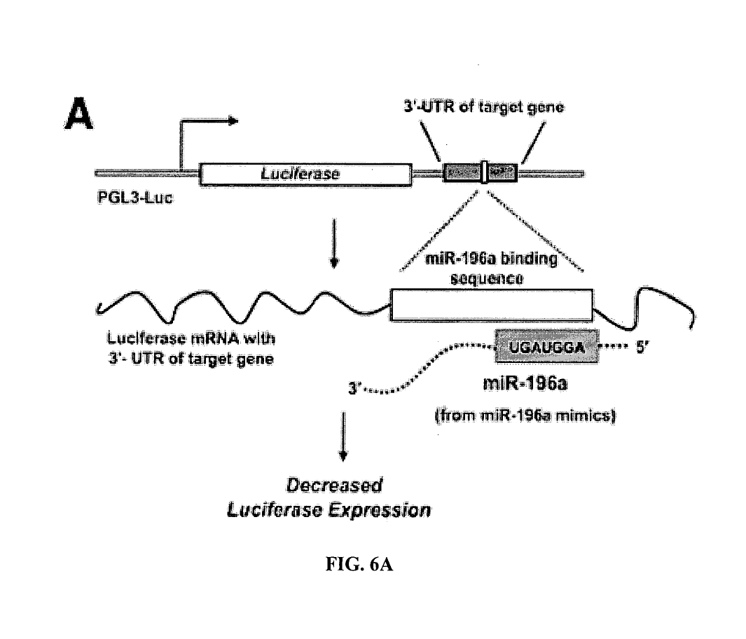

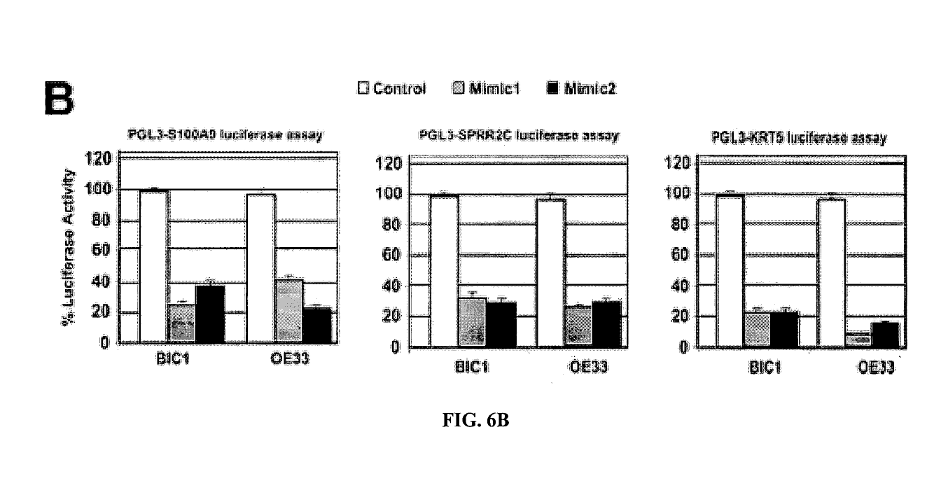

RNA mimics of miR-196a directly targets the 3′ untranslated regions (UTR) of SPRR2C, S100A9 and KRT5 mRNA. A luciferase-based assay was employed to further confirm the direct targeting of SPRR2C, S100A9 and KRT5 mRNAs by miR-196a. The 3′-UTRs of these targets which included the miR-196a binding sites was cloned (shown in FIG. 1) into a PGL3 luciferase reporter plasmid and the effect of increasing miR-196a levels on luciferase expression was tested. The schematic representation of the principle behind the assay is depicted in FIG. 6A. Results of assay done in two different esophageal cancer cell lines, BIC1 and OE33 showed that increasing the level of miR-196a consistently resulted in a reproducible and considerable decrease (60-90%) in the luciferase activity compared to the negative mimic control in all 3 genes (FIG. 6B). This confirmed that these 3 genes are direct targets of miR-196a. Similar experiments confirmed that CSTA is also a direct target of miR-196a.

### Example 2

### MicroRNA-196a Targets Annexin A1

### A microRNA-Mediated Mechanism of Annexin A1 Downregulation in Cancers

### Materials and Methods

Cell lines. The 12 cell lines included in the study were EA cell lines SEG-1, BIC-1, 0E33, and SKGT-5, breast cancer cell lines MDA-231, MDA-453, MDA-435, MCF7 and T47D, and endometrial cancer cell lines HEC-1A, HEC-1B, Ishikawa and AM3CA.

Patient samples. Institutional database at Department of Pathology, The University of Texas MD Anderson Cancer Center was searched to identify patients with EA who underwent esophagectomy without prior chemoradiation. All patients had clinically localized disease as per the endoscopic ultrasonography and imaging findings, which included positron emission tomography scan. EAs from 20 patients were included in the study. Ten of the specimens had matched gastric (six cases) or esophageal (four cases) tissue available to use as normal control tissue. Five-micron thick slides were prepared from the paraffin block and RNA was isolated following manual microdissection. All tissue specimens were collected through a protocol approved by MD Anderson Cancer Center's Institutional Review Board.

Real-time quantitative PCR for ANXA1 mRNA and miRNA expression analysis. Total RNA was extracted from the cell lines using Trizol (Gibco BRL, Life Technologies, Gaithersburg, Md., USA) as per the manufacturer's protocol. For cDNA synthesis, 100 ng total RNA from each cell line was reverse transcribed in a final volume of 20 μl using random primers and SuperScript II Reverse Transcriptase (Invitrogen, Carlsbad, Calif., USA). The Taqman minor groove binder probe and the ABI Prism 7900 HT Sequence Detection System (PE Applied Biosystems, Foster City, Calif., USA) were used for performing real-time PCR. The primers and probe for ANXA1 mRNA and an internal control glucuronidase beta (GUSB) mRNA, were obtained from PE Applied Biosystems through their Assays-on-Demand gene expression products services. PCR assays included 10 μl of Taqman Universal Master Mix No Amperase UNG (2×), 1 μl of 20× Assays-on-Demand Gene Expression Assay Mix and μl of cDNA diluted in RNase-free water, in a final volume of 20 μl. The PCR thermalcycling conditions were as follows: 10 min at 95° C. for AmpliTaq Gold activation and 40 cycles for the melting (95° C., 15 s) and annealing/extension (60° C., 1 min) steps. Each target was amplified individually and in duplicate. The relative levels of ANXA1 mRNA were calculated based on the difference between amplification of ANXA1 and GUSB mRNA using the delta CT (ΔCT) method.

RNA from formalin-fixed paraffin-embedded tissues was isolated after manual microdissection using the RecoverALL Total Nucleic Acid isolation kit (Ambion/Applied Biosystems, Austin, Tex., USA). Reverse transcription was performed as described above, except with 300 ng of total RNA. Real-time qPCRs for ANXA1 and GUSB mRNAs were performed using 5 μl of cDNA for each target.

The relative levels of miR-584, miR-196b and miR-196a were determined by stem loop real-time qPCR using gene-specific primers according to the TaqMan MicroRNA Assay protocol (PE Applied Biosystems). For reverse transcription, 7 ng of total RNA was used for cell lines, whereas 50 ng of total RNA was used for formalin-fixed paraffin-embedded samples. miR-16 was selected as the normalizer, as this miRNA showed minimal variation in expression among different cell lines and cancer specimens. Each miRNA was amplified individually and in duplicate. The relative levels of individual miRNAs with reference to miR-16 were calculated using the ΔCT method.

Western blot analysis. Cell lysates from the cancer cell lines were prepared using Beadlyte Universal Lysis buffer (Upstate, Lake Placid, N.Y., USA) with protease inhibitor cocktail (Roche Diagnostics, Indianapolis, Ind., USA). Total proteins were resolved by SDS-polyacrylamide gel electrophoresis using a 12% acrylamide gel and transferred to a nitrocellulose membrane. The membranes were probed with antibodies for ANXA1 (BD Biosciences, San Jose, Calif., USA) and β-actin (Sigma-Aldrich, St Louis, Mo., USA).

Transfection with miR-196a mimics. Two RNA mimics for miR-196a (designated as mimics 196a1 and 196a2) were purchased from Dharmacon Inc. (Chicago, Ill., USA). The mimics were transfected into cultured cells using DharmaFECT Duo transfection reagent (Dharmacon Inc.). The final concentration of the mimics was 40 nM. After 48 h, the cells were harvested to measure ANXA1 protein and mRNA levels as described above. A nonspecific miRNA mimic was used as an appropriate negative control.

Cell proliferation assay. The proliferation assay was done in a 96-well format using CellTitre 96 One solution Cell proliferation assay kit (Promega Corporation, Madison, Wis., USA). Mimics of miR-196a were transfected as described above and after 96 h, the proliferation of the cells was assayed. In an individual experiment, proliferation under each condition was studied in triplicate and the overall experiment was repeated at least twice.

Cell death assay. Twenty-four hours post-transfection with RNA mimics of miR-196a and the mimic negative control, the cells were treated with 100 nM staurosporine (Calbiochem, San Diego, Calif., USA) and the extent of cell death was estimated by trypan blue staining after 5 and 20 h using Vi-Cell cell counter (Beckman-Coulter, Fullerton, Calif., USA).

Colony-formation assay. Twenty-four hours post-transfection with RNA mimics, cells were counted and seeded in methylcellulose. After 14 days, colonies were counted and photographed under microscope.

Cloning of the 3′-UTR of ANXA1 into PGL3 vector and luciferase assay. The 284-bp 3′-UTR region of ANXA1 containing the putative miR-196a recognition site was amplified from the genomic DNA of MCF-7 breast cancer cells. The amplified fragment was then cloned into the pGL3 control vector (Promega Corporation) at the XbaI site and confirmed by sequencing. The primers used were as follows: forward sense primer, 5′-GCATCTAGAACATTCCCTTGATGGTC-3′ (SEQ ID NO:11) and reverse antisense primer, 5′-CCGCATCTAGAGTGACGTCATTTTATTTTC-3′ (SEQ ID NO:12). Cotransfection of the plasmid and the RNA mimics into the cells was accomplished using DharmaFECT Duo transfection reagent (Dharmacon Inc.). Luciferase assay was performed 48 h after transfection using the Dual Luciferase Reporter Assay System kit (Promega Corporation).

Statistical analysis. Pearson's product-moment correlation coefficient was used to measure the degree of the linear relationship between mRNA levels of miR-196a, miR-196b and miR-584 as compared to ANXA1 across the 12 cell lines. These correlations were tested for significance under the assumption that under the null hypothesis of no linear relationship, the test statistic

$${t}_{r} \sqrt{n - 2 \times } \sqrt{\frac{{r}^{2}}{1 - {r}^{2}}}$$

follows a t-distribution with (n-2) degrees of freedom when the sample correlation r is based on a sample from a bivariate normal distribution. Pearson correlation was also used to measure correlation between ANXA1 mRNA and miR-196a levels in esophageal cancers. A paired t-test was used to investigate expression level differences between normal and cancerous tissue. P-values<0.05 were considered statistically significant.

### Results

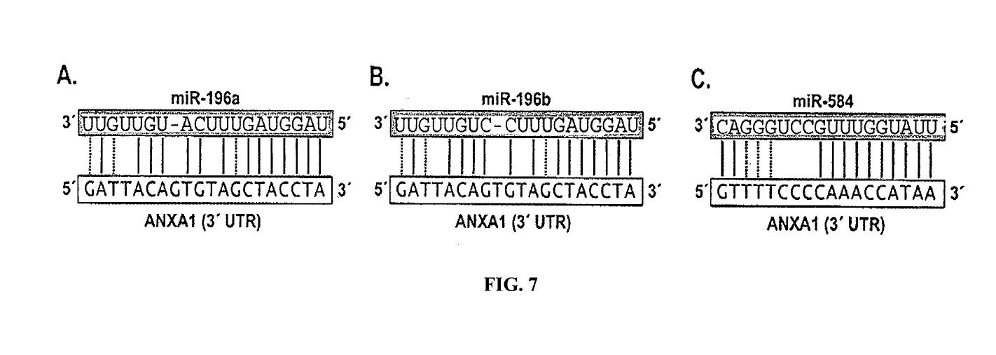

Computational analysis. The Sanger database predicts the potential miRNAs that target mRNA of any given gene by comparing the complementarity of the miRNA sequence to the 3′-UTR of the mRNA. The potential is scored depending on the extent of complementarity between the sequences at the 5′-end of the miRNA and the binding region in the 3′-UTR of the mRNA. The database predicted miR-584, miR-196a and miR-196b as potential miRNAs to target ANXA1 mRNA, with miR-584 showing the highest complementarity score. The base complementarities of these three miRNAs to the target 3′-UTR region of ANXA1 mRNA are shown in FIG. 7.

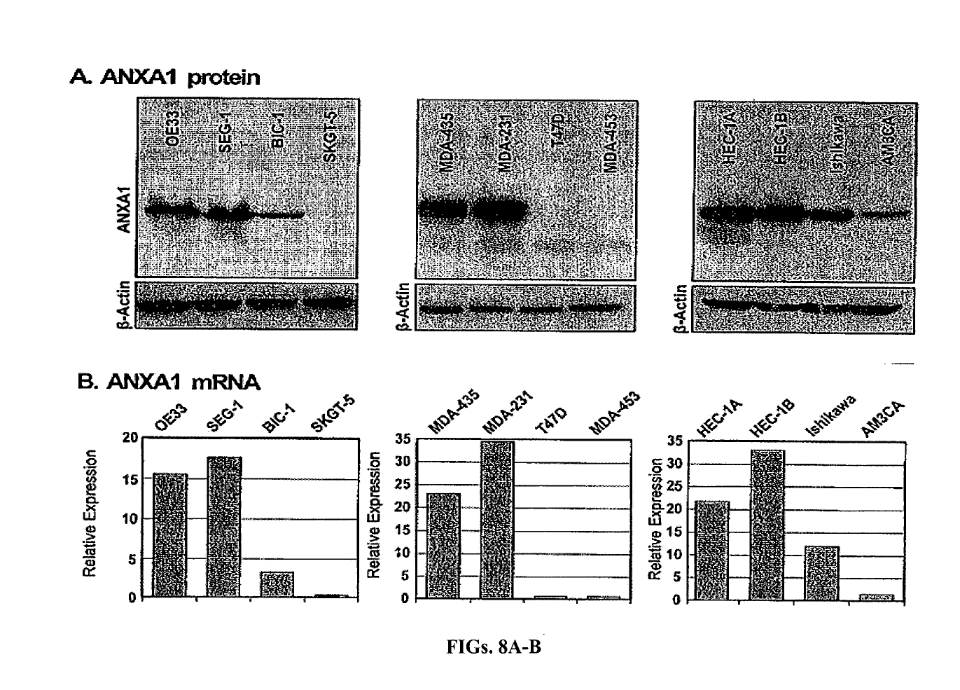

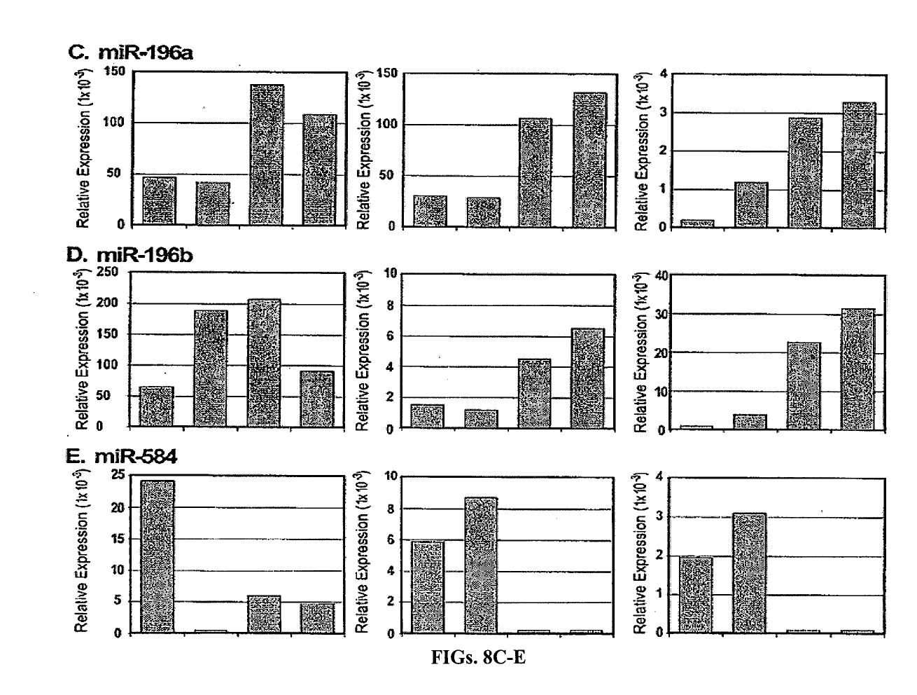

Correlation of miR-196a, miR-196b and miR-584 levels with ANXA1 expression in cancer cell lines. Western blot analysis of the 12 cell lines representing esophageal, breast and endometrial cancers revealed that ANXA1 levels varied considerably among the cell lines, with a few cell lines showing negligible ANXA1 expression (FIG. 8A). Levels of ANXA1 mRNA measured by real-time quantitative PCR (qPCR) followed the pattern seen at protein level indicating that ANXA1 mRNA levels are a good measure of the protein levels (FIG. 8B).

miR-196a levels correlated inversely with the ANXA1 mRNA levels across all the 12 cell lines tested (FIG. 8C). In the esophageal cancer cell lines OE33 and SEG-1, the ANXA1 mRNA levels were high (15.47 and 17.49, respectively), which corresponded to low miR-196a (0.046 and 0.041, respectively). In contrast, BIC-1 and SKGT-5 esophageal cancer cells had low ANXA1 mRNA (3.23 and 0.0026, respectively) and correspondingly high levels of miR-196a (0.13 and 0.11, respectively) (FIGS. 8B and 6C, left panels). The breast cancer cell lines, T47D and MDA-453, had highly suppressed ANXA1 levels (≦0.06) with comparatively high levels of miR-196a (relative expression ≧0.1), whereas MDA-231 and MDA-435 cells, which exhibited elevated ANXA1 mRNA levels (≧20.0), showed lower levels of miR-196a (relative expression ≦0.03) (FIGS. 8B and 8C, middle panels). Similarly, in endometrial cell lines HEC-1A and HEC-1B, high levels of ANXA1 mRNA (>20.0) corresponded with very low levels of miR-196a (≦0.001), whereas lower levels of ANXA1 mRNA (<12.0) in Ishikawa and AM3CA cells correlated with relatively higher levels of miR-196a (≧0.003) (FIGS. 8B and 6C, right panels), confirming mutually inverse correlation between miR-196a and ANXA1 mRNA. Pearson's correlation analysis, used to examine the correlation between miR-196a and ANXA1 mRNA in all 12 cell lines, yielded a correlation of −0.66, with a P-value of 0.019, indicating a statistically significant negative linear correlation between levels of ANXA1 mRNA and miR-196a. This was also confirmed by a simple analysis of variance linear regression model (P=0.019).

An inverse correlation was also observed between ANXA1 mRNA levels (FIG. 8B) and miR-196b (FIG. 8D). However, the correlation was not statistically significant (Pearson's correlation of −0.25, P=0.428). Even though miR-584 had the highest predictive score of the miRNA tested to potentially target ANXA1, it showed no inverse correlation with ANXA1 mRNA (FIGS. 8B and 8E), indicating that this miRNA is unlikely to target ANXA1.

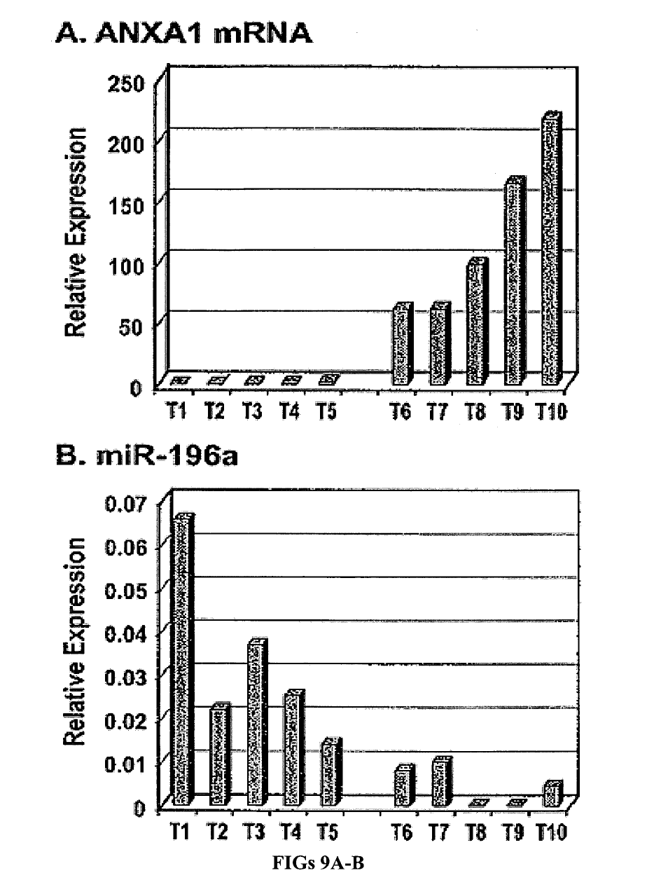

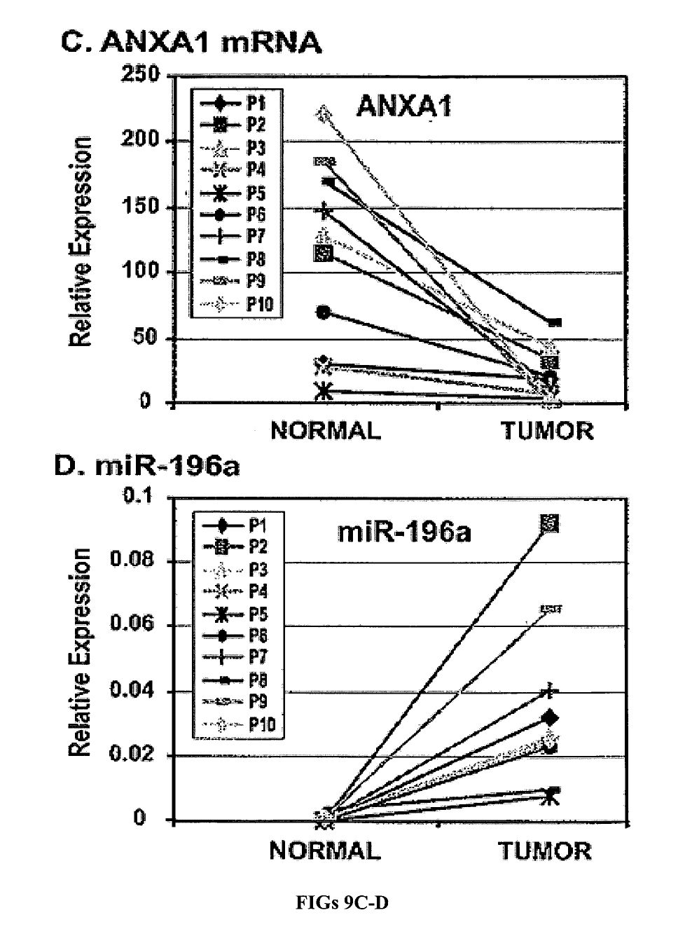

Inverse correlation between miR-196a and ANXA1 mRNA levels in esophageal cancers. To further confirm the negative correlation observed between miR-196a and ANXA1 mRNA, their levels were measured in 10 esophageal tumor tissues with varied levels of ANXA1 mRNA (FIGS. 9A and 9B). As in the cell lines, an inverse correlation was observed between ANXA1 mRNA and miR-196a level in the tumor specimens. Tumors T1 through T5 with low levels of ANXA1 mRNA showed relatively high miR-196a levels. In contrast, tumor specimens T6 through T10 with high levels of ANXA1 mRNA showed very low levels of miR-196a. The inverse correlation was statistically significant (Pearson's correlation −0.64, P=0.047).

Loss of ANXA1 expression is a frequent molecular change observed in esophageal cancer (Paweletz et al., 2000; Xia et al., 2002; Hu et al., 2004). To further confirm this observation and to investigate the possible role of miR-196a in this molecular change, the levels of ANXA1 mRNA and miR-196a were analyzed in paired normal (esophageal or gastric) and tumor tissues from 10 patients. ANXA1 mRNA levels were indeed suppressed in the esophageal tumors compared with the normal tissue (FIG. 9C, P=0.004). Interestingly, a concomitant elevation of the miR-196a levels was observed in the tumor tissue, with miR-196a levels increasing by 10- to 100-fold compared to those in corresponding normal tissue (FIG. 9D, P=0.003). This drastic increase in the tumor levels of miR-196a indicated not only a signature change of this miRNA in esophageal cancers but also the potential role of this miRNA in decreased ANXA1. In light of this inverse correlation between miR-196a and ANXA1 mRNA, further experimental efforts were focused in establishing ANXA1 mRNA as a direct target of miR-196a.

RNA mimics of miR-196a suppress ANXA1 mRNA levels in cancer cell lines. To confirm that miR-196a specifically regulates ANXA1, the gain-of-function effects of miR-196a on ANXA1 mRNA were studied. To achieve elevated levels of miR-196a in cells, two mimics of miR-196a were transfected into esophageal, endometrial and breast cancer cell lines and the consequent effect on the levels of ANXA1 mRNA was monitored by real-time qPCR and western blotting analysis. The miRNA mimics were synthetic double-stranded RNA oligonucleotides, which on delivery generated higher levels of miR-196a in the cells, thus amplifying its effects. It is of interest to note that mature miR-196a can be generated by two distinct precursor RNAs that originate from two different loci, one located on chromosome 17 and the other on chromosome 12. However, the final mature miR-196a sequence generated from both precursors is identical. To determine if the source of mature miRNA affected the target levels, we used both mimic 196a1 and mimic 196a2, which corresponded to the two different precursor sequences.

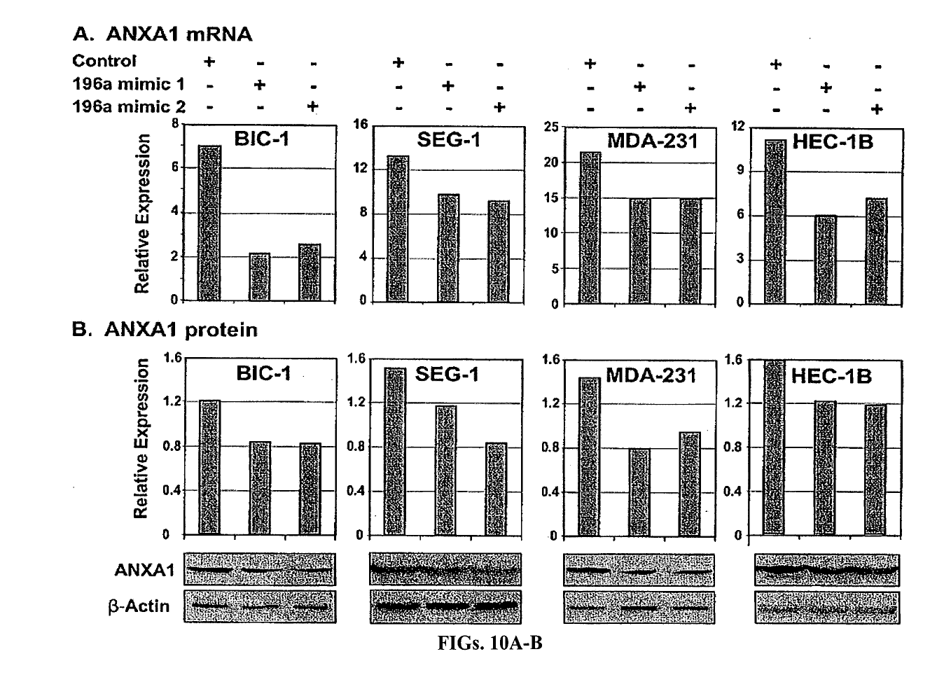

As shown in FIG. 10A, transfection of cancer cells with miR-196a mimics resulted in a reproducible decrease of ANXA1 mRNA by 60-65% in BIC-1, 30-35% in SEG-1, 30-35% in MDA-231 and 40-45% in HEC1B cells when compared to the respective controls that were transfected with a nonspecific negative control RNA. The decrease in the ANXA1 mRNA levels was accompanied by a decrease in ANXA1 protein levels in these four cell lines (FIG. 10B). On the whole, these experiments demonstrated that increasing the level of miR-196a resulted in concomitant suppression of the mRNA as well as protein levels of ANXA1 in cell lines from three different cancer types. This finding provides further evidence that ANXA1 is indeed a target of miR-196a.

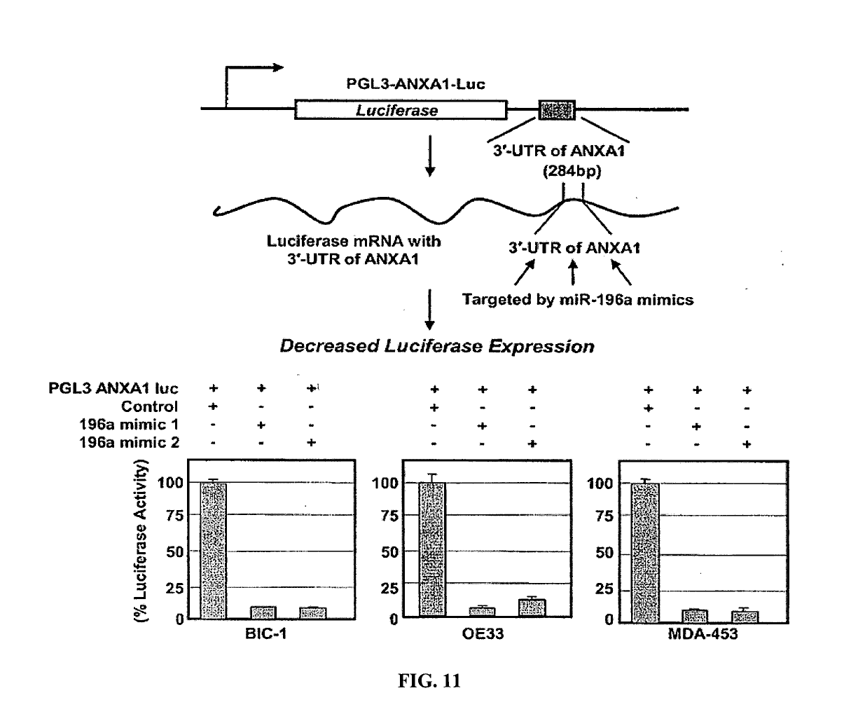

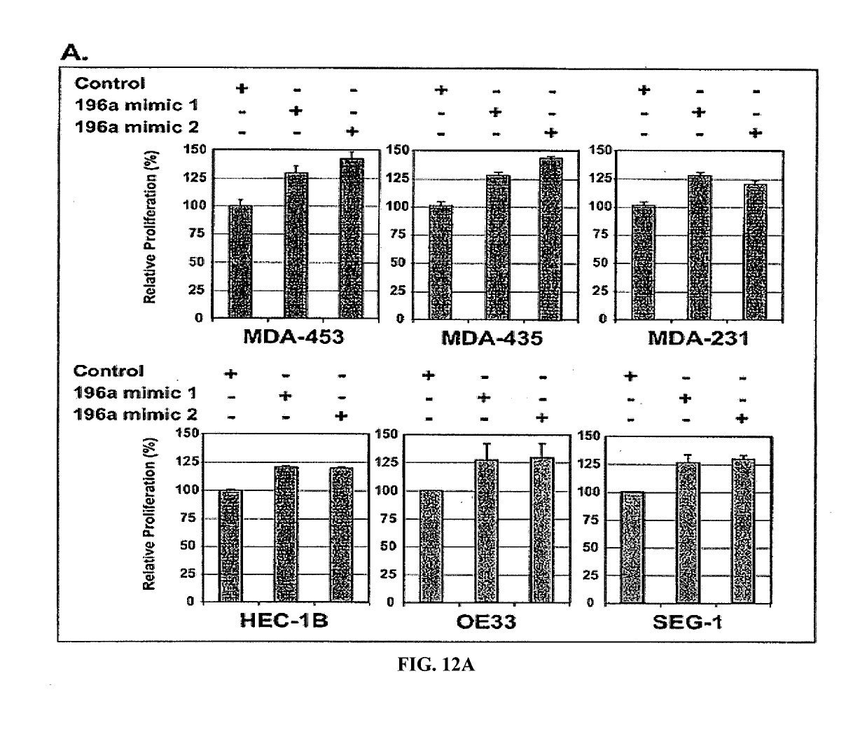

RNA mimics of miR-196a directly target the 3′-UTR of ANXA1 mRNA. To evaluate if the negative regulation of ANXA1 by miR-196a was a direct effect, a 284-bp fragment of the 3′-UTR region of ANXA1 mRNA that included the predicted miR-196a recognition site (FIG. 11A) was subcloned into a luciferase reporter plasmid designated as PGL3-ANXA1-LUC (FIG. 11, upper panel). This luciferase reporter construct was cotransfected with miR-196a1 and miR-196a2 mimics into two esophageal cell lines, BIC-1 and OE33 with high and low levels of endogenous miR-196a levels, respectively. Both mimics caused greater than 90% decrease in the luciferase activity compared to the negative mimic control (FIG. 11, lower panel). A similar suppression was also observed in the breast cancer cell line MDA-453. This experiment demonstrated clearly that miR-196a affects ANXA1 expression by directly binding to and targeting the complementary 3′-UTR region of ANXA1 and validated that ANXA1 was a bonafide target of miR-196a (FIG. 11).

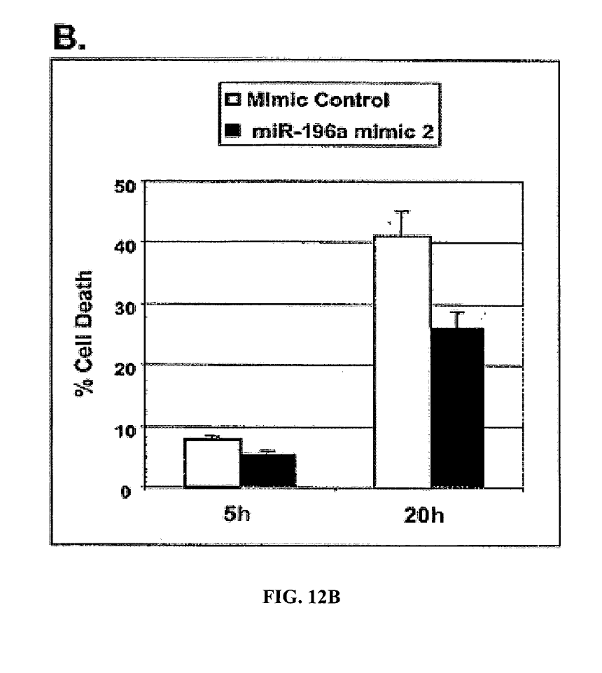

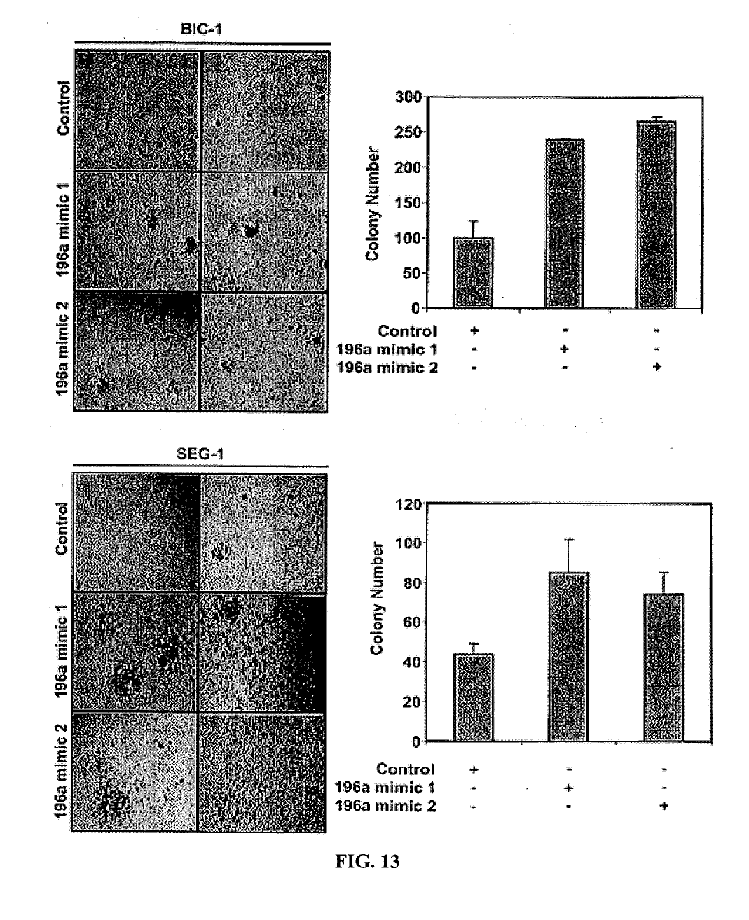

RNA mimics of miR-196a enhance cell proliferation in cancer cell lines. To test whether increased levels of miR-196a might be involved in increasing the proliferative potential of cancer cells, the effect of miR-196a RNA mimics on proliferation in six cell lines representing esophageal, breast and endometrial cancers were assessed. A consistent increase in the cell proliferation was observed in all the cell lines transfected with miR-196a RNA mimics (FIG. 12A). The two RNA mimics increased proliferation within 96 h of transfection in comparison to mimic negative control in three breast cancer cell lines MDA-453, MDA-435 and MDA-231 by 20-45% (FIG. 12A, upper panel), in two esophageal cancer cell lines OE33 and SEG-1 by 25-30% (FIG. 12A, lower panel) and in an endometrial cancer cell line HEC-1B by 20% (FIG. 12A, lower panel). This consistent trend of enhanced proliferation within 96 h after transfection of the mimics showed that miR-196a could be physiologically involved in increasing the proliferative potential of cancer cells. Consequently, the characteristic increase of miR-196a levels observed in esophageal carcinomas (FIG. 13) could be one of the important physiological changes occurring during the neoplastic transformation of normal esophageal tissue.

RNA mimics of miR-196a enhance colony-forming ability in esophageal cancer cell lines. To further characterize the effects of miR-196a on the growth characteristics of esophageal cancer cells, the effect of miR-196a overexpression on anchorage-independent growth of BIC-1 and SEG-1 was determined by examining their colony-forming ability after transfection with the mimics. At 2 weeks of growth in methylcellulose, a clear trend of increase in both the number and the size of the colonies was observed. In BIC-1 cells, the mimics boosted the colony-forming ability as evident by the increase in the colony size and colony numbers (>2-fold) (FIG. 13, upper panels). Similar increase in the colony number and more notably in colony size was observed in SEG-1 cells (FIG. 13, lower panels). Both the mimics in general exhibited similar trend of effects in our assays, with small differences (<10%) in the extent of the effects proving that an effective increase in the level of miR-196a could be achieved by employing any one of the two mimics.

RNA mimic of miR-196a shows suppression of apoptosis. To test if increased miR-196a levels result in reduced apoptosis, the effect of miR-196a overexpression on apoptosis was studied in esophageal cancer cell line SEG-1. Twenty-four hours following transfection with miR-196a mimic 2, apoptosis was induced by treating cells with 100 nM staurosporine, a potent inhibitor of protein kinase C. The extent of cell death was assayed by trypan blue staining. After 5 h of staurosporine treatment, cells transfected with the mimic showed 5% cell death compared to 8% in control. After 20 h, 25% cell death was observed in mimic-transfected cells as compared to 41% in control (FIG. 12B). This suggests that miR-196a has an antiapoptotic effect.

All of the methods disclosed and claimed herein can be made and executed without undue experimentation in light of the present disclosure. While the methods of this invention have been described in terms of preferred embodiments, it will be apparent to those of skill in the art that variations may be applied to the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,234,809
U.S. Pat. No. 5,432,272
U.S. Pat. No. 5,538,848
U.S. Pat. No. 5,614,365
U.S. Pat. No. 5,789,224
U.S. Pat. No. 5,965,364
U.S. Pat. No. 6,001,983
U.S. Pat. No. 7,001,724
U.S. Patent Publn. 20030194726
Ausubel et al., Current Protocols in Molecular Biology, Greene Publ. Assoc. Inc. & John Wiley & Sons, Inc., MA, 1993.
Ausubel et al., Current Protocols in Molecular Biology, John Wiley & Sons, NY, 1987.
Barringer, et al., Gene, 89:117, 1990.
Best et al., Applied Statistics, 24:377-379, 1975.
Bloomston et al., Jama, 297:1901-1908, 2007.
Cabral et al., J. Biol. Chem., 276:19231-19237, 2001.
Calin and Croce, Nat. Rev. Cancer, 6:857-866, 2006.
Chambers, In: Linear models. Chapter 4 of Statistical Models, Chambers and Hastie (Eds.), Wadsworth & Brooks/Cole, 1992.
Chen, N. Engl. J. Med., 353:1768-1771, 2005.
Debernardi et al., Leukemia, 21:912-916, 2007.
Devesa et al., Cancer, 83:2049-2053, 1998.
Esquela-Kerscher and Slack, Nat. Rev. Cancer, 6:259-269, 2006.
Guatelli, et al., Proc. Nat. Acad. Sci. USA, 87: 1874, 1990.
Holm, Scandinavian J. Statistics, 6:65-70, 1979.
Hornshetein et al., Nature, 438:671-674, 2005.
Hu et al., Clin. Cancer Res., 10:6013-6022, 2004.
Innis, et al., In: PCR Protocols. A guide to Methods and Application, Academic Press, Inc. San Diego, 1990.
Kimchi et al., Cancer Res., 65:3146-3154, 2005.
Kwoh et al., Proc. Natl. Acad. Sci. USA, 86:1173, 1989.
Laboratory Techniques in Biochemistry and Molecular Biology: Hybridization With Nucleic Acid Probes, Theory and Nucleic Acid Preparation, Tijssen (Ed.), Elsevier, NY, Chapt. 3 (Pt I), 1993.
Landegren et al., Science 241:1077-1080, 1988.
Luthra et al., Clin. Cancer Res., 13:912-919, 2007.
Luthra et al., J. Clin. Oncol., 24:259-267, 2006.
Miller et al., J. Immunol., 140:52, 1988.
Olsen et al., Dev. Biol., 216:671, 1999.
Patel et al., Mamm. Genome, 14:140-148, 2003.
Paweletz et al., Cancer Res., 60:6293-6297, 2000.
PCT Appln. WO 00/56748
PCT Appln. WO 00/66604
Ramaswamy et al., Proc. Natl. Acad. Sci. USA, 98:15149-15154, 2001.
Reid et al., Am. J. Gastroenterol., 96:2839-2848, 2001.
Reid et al., Gastrointest. Endosc. Clin. N Am., 13:369-397, 2003.
Rosenfeld et al., Nat. Biotechnol., 26:462-469, 2008.
Sambrook et al., Molecular cloning. A laboratory manual; 2^{nd} Ed., Cold Spring Harbor Laboratory Press, 1989.
Sato et al., Biochem. Biophys. Res. Commun., 342:465-471, 2006.
Schmitt et al., Nucleic Acid Res., 18:3091-3092, 1990.
Seggerson et al., Dev. Biol., 243:215, 2002.
Selaru et al., Oncogene, 21:475-478, 2002.
Shaheen et al., Gastroenterology, 119:333-338, 2000.
Springer, In: American Joint Committee on Cancer Staging Atlas, 6^{th} Ed., 77-89, 2006.
Swisher et al., Am. J. Surg., 169:609-614, 1995.
Tricoli and Jacobson, Cancer Res., 67:4553-4555, 2007.
Walsh et al., Biotechniques, 10(4):506-513, 1991.
Wu and Wallace, Genomics, 4:560-569, 1989.
Xia et al., Oncogene, 21:6641-6648, 2002.

1. A method for diagnosing cancer or dysplasia of the esophagus in a sample of tissue, comprising testing cells of the sample to assess whether miR-196a (SEQ ID NO:1) expression in cells of the sample is greater than a reference control miR-196a expression, wherein a greater expression of miR-196a in cells of the sample compared to the reference control is indicative of esophageal cancer or dysplasia.

2. The method of claim 1, wherein the sample of tissue is from a mammal.

3. The method of claim 2, wherein the mammal is a human.

4. The method of claim 1, wherein the sample is from a subject that has been previously diagnosed with a premalignant lesion of the esophagus.

5. The method of claim 4, wherein the premalignant lesion of the esophagus is Barrett's esophagus.

6. The method of claim 1, wherein the sample comprises esophageal mucosa.

7. The method of claim 1, further comprising determining the expression of miR-196a of the reference control.

8. The method of claim 3, wherein the reference control miR-196a expression is miR-196a expression from noncancerous human cells.

9. The method of claim 8, wherein the noncancerous human cells are normal human mucosal cells.

10. The method of claim 8, wherein the noncancerous human cells are dysplastic mucosal cells.

11. The method of claim 1, wherein the noncancerous human cells are from a subject without an esophageal lesion.

12. The method of claim 11, wherein the noncancerous human cells are squamous mucosal cells.

13. The method of claim 3, wherein the human does not have symptoms of an esophageal lesion.

14. The method of claim 3, wherein the human has symptoms of an esophageal lesion.

15. The method of claim 2, wherein the human has been previously diagnosed with an esophageal lesion.

16. The method of claim 1, further comprising performing fluorescence in situ hybridization (FISH), DNA amplification, microarray hybridization, bead-based hybridization, allele specific oligonucleotide hybridization, size analysis, sequencing, hybridization, 5′ nuclease digestion, allele specific hybridization, primer specific extension, or an oligonucleotide ligation assay on cells of the sample.

17. The method of claim 1, further comprising obtaining a sample of suspected cancerous tissue from a subject.

18. The method of claim 1, further comprising testing cells of the sample to assess whether the expression of annexin A1 (ANXA1) in cells of the sample is lower than ANXA1 expression of normal cells, wherein a reduced expression of ANXA1 in cells of the sample compared to expression of ANXA1 in normal cells is indicative of esophageal cancer.

19. The method of claim 1, further comprising testing cells of the sample to assess whether the expression of keratin 5 (KRT5) in cells of the sample is lower than KRT5 expression of normal cells, wherein a reduced expression of KRT5 in cells of the sample compared to expression of KRT5 in normal cells is indicative of esophageal cancer.

20. The method of claim 1, further comprising testing cells of the sample to assess whether the expression of small proline-rich protein 2C (SPRR2C) in cells of the sample is lower than SPRR2C expression of normal cells, wherein a reduced expression of SPRR2C in cells of the sample compared to expression of SPRR2C in normal cells is indicative of esophageal cancer.

21. The method of claim 1, further comprising testing cells of the sample to assess whether the expression of S100 calcium-binding protein A9 (S100A9) in cells of the sample is lower than S100A9 expression of normal cells, wherein a reduced expression of S100A9 in cells of the sample compared to expression of S100A9 in normal cells is indicative of esophageal cancer.

22. The method of claim 1, further comprising testing cells of the sample to assess whether the expression of cystatin-A (CSTA) in cells of the sample is lower than CSTA expression of normal cells, wherein a reduced expression of CSTA in cells of the sample compared to expression of CSTA in normal cells is indicative of esophageal cancer.

23. The method of claim 1, further comprising testing cells of the sample to assess whether the expression of myelin and lymphocyte protein (MAL) in cells of the sample is lower than MAL expression of normal cells, wherein a reduced expression of MAL in cells of the sample compared to expression of MAL in normal cells is indicative of esophageal cancer.

24. A method for diagnosing dysplasia of the esophagus in a sample of suspected cancerous tissue, comprising testing cells of the sample to assess whether miR-196a (SEQ ID NO:1) expression in cells of the sample is greater than a reference control miR-196a expression, wherein a greater expression of miR-196a in cells of the sample compared to the reference control is indicative of esophageal dysplasia.

25. The method of claim 24, wherein the reference control miR-196a expression is miR-196a expression from dysplastic mucosal cells of the esophagus.

26. A method for diagnosing esophageal cancer in a sample of suspected cancerous tissue, comprising testing cells of the sample to assess whether the expression of ANXA1, KRT5, SPRR2C, S100A9, CSTA, or MAL in cells of the sample is reduced compared to a reference control, wherein said reduced expression in cells of the sample compared to the reference control is indicative of cancer and the reference control is a reference control of ANXA1, KRT5, SPRR2C, or S100A9 expression if the expression of ANXA1, KRT5, SPRR2C, S100A9, CSTA, or MAL, respectively of the sample is tested.

27. The method of claim 26, wherein said protein is ANXA1.

28. The method of claim 26, wherein said protein is KRT5.

29. The method of claim 26, wherein said protein is SPRR2C.

30. The method of claim 26, wherein said protein is CSTA.

31. The method of claim 26, wherein said protein is MAL.

32. The method of claim 26, further comprising testing cells of the sample to assess whether miR-196a (SEQ ID NO:1) expression in cells of the sample is greater than miR-196a expression of normal cells, wherein a greater expression of miR-196a is indicative of esophageal cancer.

33. A method of classifying cells of an esophagus as normal, dysplastic, or adenocarcinoma, comprising:
(a) testing a sample of cells of the esophagus to assess miR-196a (SEQ ID NO:1) expression;
(b) comparing said miR-196a expression in the sample of cells to the expression of a first reference control miR-196a expression and a second reference control miR-196a expression;
wherein (i) a greater expression of miR-196a in the sample of cells compared to the first reference control is indicative of adenocarcinoma and (ii) an expression of miR-196a in the sample of cells that is the same or less than the first reference control but which is greater than a second reference control is indicative of dysplasia.

34. The method of claim 33, wherein the sample of cells of the esophagus are human cells.

35. The method of claim 33, wherein the sample of cells of the esophagus are esophageal mucosal cells.

36. The method of claim 33, further comprising obtaining a sample of cells from the esophagus of a subject.

37. The method of claim 33, wherein the subject has been previously diagnosed with Barrett's esophagus.

38. The method of claim 33, further comprising performing fluorescence in situ hybridization (FISH), DNA amplification, microarray hybridization, allele specific oligonucleotide hybridization, size analysis, sequencing, hybridization, 5′ nuclease digestion, allele specific hybridization, primer specific extension, or an oligonucleotide ligation assay on the sample of cells.

39. The method of claim 38, wherein the first reference control miR-196a expression is expression of miR-196a from dysplastic mucosal cells of an esophagus.

40. The method of claim 38, wherein the second reference control miR-196a expression is expression of miR-196a from normal mucosal cells of an esophagus.

41. A method of diagnosing esophageal cancer or a dysplastic lesion of the esophagus, comprising:
(a) providing a kit comprising at least one PCR primer needed to perform amplification of miR-196a; and
(b) distributing said kit to an individual desiring to diagnose esophageal cancer or a dysplastic lesion of the esophagus in a subject.

42. The method of claim 41, wherein the kit further comprises a mix of nucleotide triphosphates.

43. The method of claim 41, wherein the kit further comprises a polymerase or a reverse transcriptase.

44. The method of claim 41, wherein the kit further comprises instructions for use in methods for diagnosing esophageal cancer or diagnosing a dysplastic lesion of the esophagus.

45. The method of claim 41, wherein the primer comprises SEQ ID NO:11 or SEQ ID NO:12.
